SEQUENCE LISTING (12 sequences)

SEQ ID NO: 1 - protein, 30 aa, Artificial sequence
  derived from CXCL2
GRREEKVGKKEKIGKKKRQKKRKAAQKRKN

SEQ ID NO: 2 - DNA, 20 bp, Artificial Sequence
  primer
tgcccttcagattgttgcac

SEQ ID NO: 3 - DNA, 21 bp, Artificial Sequence
  primer
gctaactggttagggtaatac

SEQ ID NO: 4 - DNA, 21 bp, Artificial Sequence
  primer
gctttaaacaagaggctcaag

SEQ ID NO: 5 - DNA, 20 bp, Artificial Sequence
  primer
cctcctgcctcagctcaaag

SEQ ID NO: 6 - DNA, 20 bp, Artificial Sequence
  primer
tgcccttcagattgttgcac

SEQ ID NO: 7 - DNA, 35 bp, Artificial Sequence
  primer
gcgagttacaaagcgccagagcagagcgcactgcg

SEQ ID NO: 8 - protein, 9 aa, Artificial Sequence
  Tag sequence
TETSQVAPA

SEQ ID NO: 9 - DNA, 18 bp, Artificial Sequence
  primer
cccttcagattgttgcac

SEQ ID NO: 10 - DNA, 19 bp, Artificial Sequence
  primer
taactggttagggtaatac

SEQ ID NO: 11 - DNA, 19 bp, Artificial Sequence
  primer
tgagcctcttgtttaaagc

SEQ ID NO: 12 - DNA, 32 bp, Artificial Sequence
  primer
agttacaaagcgccagagcagagcgcactgcg

(19) United States
(12) Patent Application Publication
(10) Pub. No.: US 2011/0034383 A1
(43) Pub. Date: Feb. 10, 2011

(54) CXCL12 GAMMA A CHEMOKINE AND USES THEREOF

(75) Inventors: Fernando Arenzana, Meudon (FR); Hugues Lortat-Jacob, Saint Ismier (FR); Francoise Baleux, Paris (FR)

Correspondence Address: YOUNG & THOMPSON, 209 Madison Street, Suite 500, Alexandria, VA, 22314, US

(73) Assignee: INSTITUT PASTEUR, Paris Cedex 15 (FR)

(21) Appl. No.: 12/682,943

(22) PCT Filed: Oct. 15, 2008
(86) PCT No.: PCT/IB2008/003838

§ 371 Date: Oct. 18, 2010

Related U.S. Application Data
(60) Provisional application No. 60/979,984, filed on Oct. 15, 2007.

(51) Int. Cl. A61K 38/18; A61K 38/02; C07K 14/00; C07K 16/24; G01N 33/68; C12N 5/16; A61P 9/00

(52) U.S. Cl. 514/8.1; 514/1.1; 514/9.6; 514/7.6; 514/9.1; 530/350; 530/389.2; 530/388.23; 436/86; 435/326

(57) ABSTRACT

Fragments of CXCL12 Gamma A chemokine having improved chemotaxis activity in vivo defined by an unprecedented capacity to associate and immobilise on extracellular glycans.

The file of this patent contains at least one drawing executed in color. Copies of this patent with color drawings will be provided by the Patent and Trademark Office upon request and payment of the necessary fee.

### BACKGROUND OF THE INVENTION

1. Field of the Invention

The present invention relates to fragments of CXCL12 Gamma A chemokine having improved chemotaxis and haptotactic activity in vivo defined by an unprecedented capacity to associate and immobilise on extracellular glycans.

2. Description of the Related Art

CXCL12α, a chemokine that importantly promotes the oriented cell migration and tissue homing of many cell types, regulates key homeostatic functions and pathological processes through interactions with its cognate receptor (CXCR4) and heparan sulfate (HS). The alternative splicing of the cxcl12 gene generates a recently identified isoform, CXCL12γ, which structure/function relationships remain unexplored. The high occurrence of basic residues that characterize this isoform suggests however that it could feature specific regulation by HS.

### SUMMARY OF THE INVENTION

The invention is based on the discovery of and characterization of CXCL12 gamma as a chemokine having improved chemotaxis and haptotactic activity in vivo defined by an unprecedented capacity to associate and immobilise on extracellular glycans.

The CXCL12γ chemokine arises by alternative splicing from Cxcl12 and binds CXCR4. CXCL12γ is formed by a protein core shared by all CXCL12 isoforms, extended by a distinctive carboxy-terminal (C-ter) domain.

We show that CXCL12γ is expressed in vivo with a pattern that suggests differential regulation respect to other CXCL12 isoforms. We found that CXCL12γ displays for heparan sulfates (HS) glycosaminoglycans the highest affinity reported for a chemokine (Kd 0.9 nM). Mutagenesis experiments show that this property relies in the presence of four canonical HS-binding sites located at the C-ter domain. The C-ter domain represents a functional entity per se capable of conferring full HS-binding capacity to CXCL12γ.

In contrast to other CXCL12 isoforms, CXCL12γ remains mostly adsorbed on cell membranes upon secretion. Despite reduced agonist potency on CXCR4, the sustained binding of CXCL12γ to HS enables it to promote in vivo leukocyte attraction and angiogenesis with much higher efficiency than CXCL12α.

In good agreement, CXCL12γ mutants selectively devoid of HS-binding capacity have a dramatically reduced capacity in promoting haptotactic tissue homing of leukocytes and endothelial cell precursors, although they activate CXCR4 as potently as CXCL12α. We conclude that CXCL12γ features unique structural and functional properties that make it the paradigm of haptotactic proteins, which regulate essential homeostatic functions by promoting directional migration and selective tissue homing of cells.

In one aspect, the invention provides with a composition comprising an haptotactic homing molecule and any protein that thus remain immobilized in order to induce or regulate locally: (a) the attraction an homing of cells, (b) growth and/or differentiation of resident cells and/or (c) activation of a resident pool of cells.

In one aspect of the invention there is provided a composition of a CXCL2α, CXCL2β and/or CXCL2γ chemokine and a molecule of interest.

Examples of such molecule of interest include VGEF, EGF, Neurotrophins, NGF, FGF and others.

In one aspect of the invention there is provided a composition of a haptotactic homing molecule and a molecule of interest wherein the homing molecule is a molecule comprising a polypeptide of formula [BBXB]n wherein B is a basic aminoacid selected among arginine or lysine or histidine, X is any other amino acid and n is an integer comprised between 2 and 5 and preferably n is 4.

Examples of such haptotactic homing molecules are fragments of the C-terminal CXCL2γ (or a variant).

Compositions may comprise both molecules (haptotatic homing and interest) in a simple association and are preferably administered simultaneously. Preferably the molecules in the composition are covalently associated and can be prepared either by chemical covalent coupling (with or without spacers) or by genetic engineering by using hybrid polynucleotide sequences encoding for chimeral combined molecule)

In one aspect of the invention there is provided a molecule comprising a polypeptide of formula BBXB wherein B is a basic aminoacid selected among arginine, lysine or histidine, X is any other amino acid and n is an integer from 2 to 5. Preferably n is 4.

In one aspect of the invention there is provided a molecule comprising the amino sequence GRREEKVGKKEKIGKKKRQKKRKAAQKRKN (SEQ ID NO: 1), variants of this sequence such as those comprising the core sequence that enables the haptotatic homing activity described herein, preferably having at least the amino acid sequence of at least two BBXB motifs as well as the whole basic charge of the molecule.

Variants are polypeptide sequences that present at least 90%, better at least 95% identity with the C′-terminal CXCL2γ, that comprise at least two [BBXB]n motifs and present a high basic overall charge.

Polynucleotides encoding these amino acid sequences, vectors, host cells and methods of producing the polypeptide(s) is/are also included.

In one aspect of the invention there is provided antibodies directed to CXCL2gamma, namely specific for the C-Terminal fragment as described herein.

In one aspect of the invention there is provided a method to treat a patient wherein compositions described herein are used to facilitate delivery of one or more therapeutic agents to a patient. Treatments of pathologies include, e.g., peripheral and cardiac ischemic pathologies (ie, myocardial infarction, occlusive arterial diseases like the Buerger syndrome) requiring angiogenesis/revascularisation for maintaining physiological functions.

Moreover, therapeutic usage includes the reparation of tissues congenitally abnormal, or irreversible damaged following ischemia or degenerative processes, on the basis of the unchallenged capacity of CXCR4/CXCL12 couple to promote directional migration and tissue homing of a number of cell precursors, among which: neurons, fibroblasts, epithelial and muscular cells.

Particular therapeutic usages also include use of combination of the haptotactic homing molecules according to the invention with: (a) VEGF to treat angiogenesis related pathologies, (b) neutrophins to treat cicatrisation associated pathologies, (c) NGF to treat nerve growth associated pathologies, (d) FGF to treat pathologies implying fibroblasts default, angiogenesis and/or tissue repair.

### BRIEF DESCRIPTION OF THE DRAWINGS

FIG. 1 Tissue expression of γ-wt in human and mouse. (A) Specific immunodetection of γ-wt in HEK-293T cells. Cells were transfected either with γ-wt or β-wt pcDNA3.1 expression vectors, treated with brefeldin and labeled with the 6E9 mAb. (B) Mutagenesis of K78E79K80 in γ-wt C9 (γ-C9^{up}) prevents recognition of the chemokine by the 6E9 mAb. Western blot analysis of chemically synthesized α-wt and γ-wt (synt) and α-wt C9, γ-wt C9, γ-C9^{up} and γ-C9^{dw} C9-tagged chemokines expressed from SFV-vectors in BHK cells. L, cell lysates; S, culture supernatants. MW, molecular weight. (C) Expression of Cxdl12α and Cxdl12γ mRNAs by RT-PCR in different adult mouse tissues. RT+/−denotes presence or absence of RT enzyme. (D) Detection of CXCL12 isoforms either with K15C mAb (panCXCL12 isoforms) or anti-γ-wt 6E9 mAb in mouse and human tissues. Panel 1. Mouse adult heart. LA, left auricle; LV, left ventricle; RV, right ventricle; IVS, interventricular septum; ca, carotid artery; my, mitral valve (4×). Panel 2. Detail of a lung bronchiol (mouse E16.5 embryo, 40×). Panel 3. Mouse E16.5 embryo intestine and bladder. White arrowheads, bladder epithelium; black arrowheads, large intestine; arrows, peritoneum (10×). In inset, details of intestinal mucosa labeling (4×). Panel 4. Large abdominal vessel (mouse E16.5 embryo, 20×). Panel 5 Immunolabeling of a human inflammatory synovial tissue (rheumatoid arthritis). White arrowheads, blood vessel; black arrowheads, lining synoviocytes; arrows, fibroblasts (400×).

FIG. 2 Immobilized GAG-binding activity of γ-wt. (A) Sequence alignment of wt (γ-wt) and mutated derivatives (γ-m1 and γ-m2) of CXCL12γ protein. In bold, identified and putative HS-binding motifs; underlined, mutated amino-acids. (B) Chromatography affinity values obtained from a Heparin affinity column elution. (C) Binding of α-wt, γ-wt, γ-m1 and γ-m2 to on chip-immobilized heparin (HP). Chemokines were injected over HP activated surface for 5 min, after which running buffer was injected, and the response in RU was recorded as a function of time. Each set of sensorgrams was obtained with α-wt at (from top to bottom) 200 to 0 nM or γ-wt, γ-m1 and γ-m2 at 25 to 0 nM.

FIG. 3 Cell surface GAG-binding activity of α-wt and γ-wt on parental (K1), GAG-mutant (pgsD677, pgsA745) CHO cell lines and primary human-microvascular endothelial cells (HMVEC). Binding of α-wt or γ-wt was detected with the anti-CXCL12 K15C mAb.

FIG. 4 Electrophoretic mobility and secretion pattern of γ-wt and α-wt chemokines. (A) Western blot analysis of SFV-infected BHK cell lysates revealed by the pan anti-CXCL12 K15C mAb. Abbreviations like in FIG. 1b. Formation of dimeric forms are observed for α-wt synt, α-wt C9, γ-wt synt and γ-wt C9. (B) ELISA quantification of α-wt C9 and γ-wt C9 accumulated in the cell supernatant (S) or in the cell lysates (L) of HEK-293T cells transfected with the corresponding pcDNA3.1 expression vector. Four hours before collection, fresh medium was added and cells were treated with brefeldin (left panel) or left untreated (right panel). (C) The secreted γ-wt C9 protein revealed by the anti-C9 1D4 mAb accumulates massively at the cell surface of HEK-293T cells treated like in b), and permeabilised (left panel) or not (right panel) with saponin. In inset, cell surface CXCR4 expression of HEK-293T cells. (D) The γ-wt C9 chemokine is released from intact cells upon exposure to strong ionic force. BHK cells were infected with SFV-infectious particles driving the expression either of α-wt C9 or γ-wt C9. Thereafter, the proteins were detected by western blot analysis in the cell culture supernatant (S), the wash fluid (WF) or the cell lysate (L), upon brief exposure of cells either to PBS or hypertonic NaCl μM (NaCl).

FIG. 5 Cell signalling through CXCR4 induced either by α-wt, γ-wt or derivative chemokines. (A) [³⁵S]GTPγS binding to membranes from lymphoblastoid A3.01 T cells. (B) Chemotaxis of A3.01 cells or primary CD4⁺ T lymphocytes.

FIG. 6 Intraperitoneal recruitment of leukocyte subpopulations induced by α-wt, γ-wt and derivatives. (A) After 6 hr or (B) 15 hr of induction. In inset, total number of recovered cells. Results (mean±SD) are representative of three independent experiments.

FIG. 7 Angiogenic effect of α-wt or γ-wt and derivatives chemokines. Matrigel® containing 10 nM of each chemokine were subcutaneously injected and analyzed at day 10 from implantation. Data are representative of three independent experiments. (A) Haematoxylin-eosin staining of Matrigel® plugs. In inset, number of migrated cells (±SD) into Matrigel®. Framed, vessel-like structures forming around a central lumen. (B) Immunofluorescent detection of PECAM-1+ endothelial cells in Matrigel® neovessels with DAPI nuclear counter-labelling.

FIG. 8 Comparative GAG-binding activity of α-wt and γ-wt on parental CHO-K1 cells. Prior to incubation with the proteins, cells were treated with 10⁻³ units/mL of Heparinase (25° C.), Heparitinase I (37° C.) or Chondroitinase ABC (37° C.) degrading enzymes (Seikagaku corporation, Tokyo, Japan) for 90 minutes. Binding to control untreated cells were arbitrary set to 100 and binding observed for enzyme-treated cells was expressed as a function of signal obtained in control conditions. In inset, HS detection at the cell surface of control (K1) or Heparitinase I treated (K1+HT) CHO parental cells.

FIG. 9. CXCL12γ has an unstructured C-terminal domain but is identical to CXCL12α in the 168 region. (A) Sequences of the wild type and mutant CXCL12α, β and γ isoforms produced and used in this study (mutated residues are underlined). The secondary structures of CXCL12γ 1-68 domain and CXCL12α are almost identical (black boxes: α helices, white arrows: β strands, E: extended conformation). (B) ¹⁵N-HSQC spectrum of CXCL12γ (1 mM, 30° C.). Non overlapping amide protons assignments are indicated. Residues from the γ extension are clustered between 8-8.5 ppm ¹H frequency. (C) CXCL12γ 1-68 domain and CXCL12α fold similarly. A good correlation (Chi²=74) is observed between N—H^{N} RDCs (CXCL12γ 10-64) with RDCs backcalculated from CXCL12α structure. (D) ¹⁵N-¹H heteronuclear Noes, longitudinal (R1; square) and transversal (R2; triangle) relaxation rates on CXCL12γCXCL12γ is folded between residues 10 and 64 and the γ extension is disordered with low NOe, R2 and R1 values.

FIG. 10. Analysis of CXCL12 binding to HP, HS and DS. SPR sensorgrams measured when CXCL12 were injected over HP, HS or DS activated sensorchips. The response in RU was recorded as a function of time for CXCL12α (26 to 300 nM), β (13 to 150 nM) and γ (2.6 to 30 nM).

FIG. 11. The interaction of CXCL12γ with dp4 and dp8 HP derived oligosaccharides reveals two main binding sites. (A) Weighted chemical shift differences √((ΔδH)²+(ΔδN/10)²) of CXCL12γ (0.2 mM) amide protons upon addition of dp4 (0.56 mM magenta) and dp8 (0.5 mM blue). Unassigned amide protons are left blank. Both the core structure and the C-terminus of CXCL12γ are affected upon oligosaccharide interaction. (B) ¹⁵N-HSQC in the 1-68 domain of CXCL12γ at 0 (black), 0.13 (red) 0.3 (green) and 0.5 mM (blue) dp4 concentration. (C) ¹⁵N-HSQC of CXCL12γ C-terminal residues at 0 (black) and 0.5 mM (blue) dp4 concentration. Highly overlapped C-terminal assignments could not be all followed upon interaction. It is nevertheless obvious that most residues are perturbed upon interaction with HP derived oligosaccharides. (D) Residues 69-98 of CXCL12γ where randomised by Simulating Annealing and manually attached to CXCL12α structure (PDB 1VMC). HP dp4 structure (extracted from PDB 1HPN) is also shown. Chemical shift variations upon dp4 addition are represented on CXCL12γ in yellow, orange, or red (respectively >0.03, 0.04 or 0.08 ppm) and dark grey (not determined). A continuous binding surface is formed on CXCL12γ core domain between R20 and R41 and the last 15 residues of the protein are highly affected by the interaction.

FIG. 12. Analysis of wild type and mutant CXCL12 binding to immobilized GAGs. Binding of wild type and mutant CXCL12 were recorded as in FIG. 2. CXCL12α (26 to 300 nM), β, β-m1, β-m2 (13 to 150 nM), γ, γ-m1, γ-m2 (2.6 to 30 nM) were injected over the GAGs activated sensorchips and the response in RU was recorded as a function of time.

FIG. 13. Association and dissociation rate constant of the CXCL12-GAG interaction. (A) Graphical summary of the data generated from the sensorgrams of FIG. 4 in which association (kₒₙ) and dissociation (k_{off}) rate constants of CXCL12α (open circle), β (grey circle), β-m1 (grey square), γ

(black circle) and γ-m1 (black square) for HP were determined as described. Differences were essentially observed along the k_{off} axis. (B) Dissociative half live of the different CXCL12/HP complexes.

FIG. 14. Flow cytometric analysis of CXCL12 interaction with cell surface GAGs. CHO-K1 parental cells (squares) or HS-deficient CHO-pgsD677 cells (triangles) were incubated with the indicated concentrations of CXCL12 a (open symbols) or γ (close symbols) and, after extensive washing to remove free chemokine, were labelled with K15C mAb and analyzed by flow cytometry.

FIG. 15. Comparative analysis of the CXCR4 binding and signaling properties of CXCL12α and γ. (A) ¹²⁵I-CXCL12α (0.25 nM) was bound to CXCR4⁺ CEM cells in the presence of cold CXCL12α (squares), γ (triangle) or γ-m1 (circle). (B) Intracellular calcium mobilization induced by CXCL12α (squares), γ (triangles) isoforms or CXCL12α P2G (line) in A3.01 cells. CXCL12α P2G is a non signaling mutant of CXCL12α. Data are representative of three independent experiments.

### DETAILED DESCRIPTION OF THE PREFERRED EMBODIMENTS

Using surface plasmon resonance and NMR spectroscopy, as well as chemically and recombinantly produced chemokines, we show here that CXCL12γ first 68 amino acids adopts a structure closely related to the well described a isoform, followed by an unfolded C-terminal extension of 30 amino acids. Remarkably, 60% of these residues are either lysine or arginine, and most of them are clustered in typical HS binding sites. This provides the chemokine with the highest affinity for HS ever observed (Kd=0.9 nM), and ensures a strong retention of the chemokine at the cell surface. This was due to the unique combination of two cooperative binding sites, one strictly required, found in the structured domain of the protein, the other one being the C-terminus which essentially functions by enhancing the half life of the complexes. Importantly this peculiar C-terminus also regulates the balance between HS and CXCR4 binding, and consequently the biological activity of the chemokine.

Together these data describe an unusual binding process that gives rise to an unprecedented high affinity between a chemokine and HS. This shows that the γ isoform of CXCL12, which features unique structural and functional properties, is optimized to ensure its strong retention at the cell surface. Thus, depending on the chemokine isoform to which it binds, HS could differently orchestrate the CXCL12 mediated directional cell kinesis.

The CXCL12γ chemokine arises by alternative splicing from Cxcl12 and binds CXCR4. CXCL12γ is formed by a protein core shared by all CXCL12 isoforms, extended by a distinctive carboxy-terminal (C-ter) domain. We show that CXCL12γ is expressed in vivo with a pattern that suggests differential regulation respect to other CXCL12 isoforms. We found that CXCL12γ displays for heparan sulfates (HS) glycosaminoglycans the highest affinity reported for a chemokine (K_{d} 0.9 nM). Mutagenesis experiments show that this property relies in the presence of four canonical HS-binding sites located at the C-ter domain. In contrast to other CXCL12 isoforms, CXCL12γ remains mostly adsorbed on cell membranes upon secretion. Despite reduced agonist potency on CXCR4, the sustained binding of CXCL12γ to HS enables it to promote in vivo leukocyte attraction and angiogenesis with much higher efficiency than CXCL12α. In good agreement, CXCL12γ mutants selectively devoid of HS-binding capacity lack in vivo activity although they activate CXCR4 as potently as CXCL12α. We conclude that CXCL12γ features unique structural and functional properties that make it the paradigm of haptotactic proteins, which regulate essential homeostatic functions by promoting directional migration and selective tissue homing of cells.

The invention represents a significant advance regarding the therapeutic potential of CXCL12 and renders the isoform gamma as the paradigm of haptotactic proteins. The unchallenged capacity of this isoform to attract cells in vivo makes it the best candidate for modulating important physiopathological and homeostatic process such as the migration of progenitor cells into discrete anatomic sites. Moreover, the identification and characterisation of the distinctive carboxy-terminal domain of this protein as a key element for the biological properties of the chemokine opens the way for transferring (in cis) the outstanding affinity for heparan sulfates to other proteins (ie, chemokines, cytokines) thus improving their capacity to mediate their biological effects in a restricted and selected area. The disordered structure of this domain would facilitate the development of chimeric functional proteins.

In particular, in certain aspects of the invention the characterisation of the protein with the highest haptotactic capacity in vivo yet described could not have been expected from what was known about CXCL12 before the work described herein. Moreover, the characterisation of the protein domain responsible for the distinctive properties of the chemokine was not known nor could have been predicted based on the information available to date.

The interest of using the most potent natural attractant of progenitor cells, CXCL12, is hampered by the inability of the protein to remains localized at the discrete sites where migration and settling of cell precursor is targeted. This problem can be circumvented by the novel isoform which displays a long-lasting attachment to the extracellular matrix and induces robust and sustained biological effects. Thus, the inventions described herein advance the state significantly and to the best of the Inventors knowledge differ quite significantly from the existing technologies for obtaining such effects.

The long-lasting biological effects observed upon injection indicate that the protein in the natural, mature form is appropriated for preclinical usage. The extreme high degree (99%) of conservation of this circulating, soluble protein across the species announces a very poor antigenicity. The usage of homologous proteins should limit the already reduced risks of immunogenicity. The protein has been proved to be functional both in vivo and in vitro when produced from an expression vector.

Thus, the discoveries described herein can have direct industrial and real-world applications such treating diseases with arterial occlusive pathologies and wound healing that could benefit from induced angiogenesis and de novo formation of vessels. Beside this, the invention have direct application in the attraction and homing of cells, specialized or not, that are required for both the histologic and functional restoration of a number of tissues: myocardium, muscles, neuronal pattern where based on the outstanding capacity of CXCL12 to promote both directional migration and tissue homing of cells.

Particular therapeutic usages also include use of combination of the haptotactic homing molecules according to the invention with: (a) VEGF to treat angiogenesis related pathologies, (b) neutrophins to treat cicatrisation associated pathologies, (c) NGF to treat nerve growth associated pathologies, (d) FGF to treat pathologies implying fibroblasts default, angiogenesis and/or tissue repair.

In another aspect of the invention, beyond the applications directly linked to the usage of the CXCL12 gamma isoform, soluble proteins, such as, for example, cytokines and chemokines fused to the C-terminal domain could show enhanced physiological properties in a defined tissue environment.

Using recombinant and synthetic CXCL12 gamma proteins selectively mutated in the BBXB motif located in the core of the protein, we have recently confirmed the role per se of the carbox-terminal domain in conferring the exceptional affinity for heparan sulfates we have described. Both biochemical and biological data support this assertion. This finding reinforces our hypothesis that this domain of the protein could provided when transferred in cis to unrelated proteins the ability to bind tightly to heparan sulfates that in vivo could lead to a prolonged immobilisation of the protein in tissues

Compositions of a homing chemokine and a molecule of interest. In one aspect of the invention the homing chemokine is CXCL2alpha, CXCL2beta and/or CXCL2gamma Inclusive of those having at least 90 and/or 95% identity to the full-length sequences known and/or described herein.

The molecule of interest can be VGEF, EGF, Neurotrophins, NGF, FGF and others.

In one aspect of the invention the homing molecule is a molecule comprising a polypeptide of formula [BBXB]n wherein B is a basic aminoacid selected from arginine and/or lysine and/or histidine, X is any other amino acid and n is an integer from 2 to 5. In one embodiment, n is 4.

The common amino acids for X include, Alanine; Arginine; Asparagine; Aspartic; acid; Cysteine; Glutamic acid; Glutamine; Glycine; Histidine; Isoleucine; Leucine; Lysine; Methionine; Phenylalanine; Proline; Serine; Threonine; Tryptophan; Tyrosine; and Valine.

The haptoptatic homing molecule can be a fragment of the C-terminal CXCL2gamma (or a variant as described herein).

In one aspect of the invention, the molecule comprises the amino acid sequence GRREEKVGKKEKIGKKKRQKKRKAAQKRKN (SEQ ID NO: 1).

Inclusive of those having at least 90 and/or 95% identity to the full-length sequences known and/or described herein.

The molecules and therefore their amino acid sequence structure can be obtained from naturally sources (isolated there from), recombinantly derived or generated, and/or synthetically generated according to well-known procedures for producing synthetic molecules.

The molecules that can be employed in the inventive methods described herein are those full length coding sequences, protein sequences, and the various functional variants, chimeric proteins, muteins, and mimetics, for example PEGylated forms or albumin-coupled forms.

The variants of the sequences should share the common structural features of at least two BBXB motifs as well as maintaining the basic charge of the molecule with the haptotatic homing activity described herein.

To measure the biological activity of the assays described herein, singularly or in combination can be used.

Compositions may comprise both molecules (homing and interest) in a simple association but must be administered simultaneously.

Polynucleotides encoding one or more of the polypeptides described herein may also be constructed and used. Cloning polynucleotide fragments, generating fragments by amplification reactions such as PCR and synthetic polynucleotide construction is known in the art.

The polynucleotides can be carried on a vector or plasmid. Such vector or plasmid may also include selection markers as well as sequences to facilitate expression of the cloned polynucleotide.

The polynucleotides may also be carried in a host cell, such as human, mammalian, bacterial, fungal, insect, and others.

An example of such a plasmid is contained in the deposit at the CNCM under accession number I-3846 (pcDBACSCL12gamma) and that can express CXCL2gamma.

Preferably the molecules in the composition are covalently associated and can be prepared either by chemical covalent coupling (with or without spacers) or by genetic engineering by using hybrid polynucleotide sequences encoding for chimeral combined molecule)

Antibodies directed to CXCL2γ as well as the variants described herein, including the C-Terminal fragment can be generated using conventional techniques in the art for generating antibodies, polyclonal or monoclonal, as well as hybridomas.

The compositions described herein, having the common characteristic of the homing molecule or chemokine can be used for therapeutic treatment regimens.

In certain aspects of the invention, pathologies that can be treated include angiogenesis related pathologies such as occlusive arterial diseases.

To this regard, some example where the invention can be applied are provided: ischemic pathologies affecting extremities (ie, Buerger's syndrome) or causative of cardiovascular pathologies (ie, coronary occlusion and the subsequent myocardial infarction). The therapeutic benefit of the invention application can extend in some cases to the regeneration of tissues irreversibly damaged by the ischemia (neuronal cells, muscle).

Particular therapeutic usages also include use of combination of the haptotactic homing molecules according to the invention with: (a) VEGF to treat angiogenesis related pathologies, (b) neutrophins to treat cicatrisation associated pathologies, (c) NGF to treat nerve growth associated pathologies, (d) FGF to treat pathologies implying fibroblasts default, angiogenesis and/or tissue repair.

The CXC chemokine, stromal cell-derived factor 1/CXCL12¹ is a constitutive and broadly expressed chemokine. Mouse and human CXCL12α, the major CXCL12 isoform, differs by a single, homologous substitution (Val18 to Ile18)^{1,2} and each protein owns the capacity to bind and activate the orthologue G-protein coupled receptor (GPCR) CXCR4³. The exceptional conservation of both CXCR4 and CXCL12 structure and function in mammalians announces the essential roles played by this singular couple. CXCL12 is unique among the family of chemokines as it plays non-redundant roles during embryo life in the development of both cardiovascular⁴ and central nervous system^{5,6}, hematopoiesis⁷ and colonization of the gonads by primordial germ cells⁸. In the post-natal life, CXCL12 is involved in trans-endothelial migration of leukocytes⁹⁻¹² and regulates critically both the homing and egress of CD34+ CXCR4+ progenitor cells from the bone marrow (BM), and their migration into peripheral tissues¹³. CXCL12 plays also a prominent role in physiopathological processes such as inflammation¹⁴, angiogenesis and wound healing^{15,16}. Moreover, CXCL12 is a critical factor for growth, survival and metastatic dissemination of a number of tumors¹⁷.

The engagement of CXCR4 by CXCL12 triggers the activation of heterotrimeric Gαβγ-proteins, which ultimately promote the directional migration of cells towards a concentration gradient of ligand that defines the haptotactic function of chemokines. In vivo, chemokines form gradient concentrations by binding to glycosaminoglycans (GAG), the glycanic moieties of proteoglycans, and in particular to heparan sulfate (HS). Electrostatic contacts between the negatively charged HS and basic residues exposed at the surface of chemokines, along with structural features of the oligosaccharide, determine both the affinity and the specificity of the molecular interactions that are supposed to modulate the in vivo biological activity of chemokines complexed to proteoglycans¹⁸⁻²¹.

The study of the well characterized CXCL12α and β isoforms brought most of the knowledge of CXCL12 biological properties including interaction with GAG. In contrast, the novel CXCL12γ remains largely unexplored regarding organ and tissue expression, structure and function. CXCL12γ is formed by a core domain encompassing the 68 amino-acids of the major CXCL12α isoform shared with all CXCL12 proteins, which is extended by a carboxyterminal (C-ter) region. This region is highly-enriched in basic amino-acids and encodes four overlapped HS-binding motifs and shows identical sequence in human, rat and mouse species^{2,22,23}. We speculated that this large and charged domain could enable CXCL12γ with distinct structural and biological capacities that might determine a different ability to bind and activate CXCR4, as compared to other isoforms. Furthermore, since this domain encompasses four overlapped BBXB canonical HS-binding motifs (B for basic amino-acids, X any other amino-acid), we thought that this isoform could exhibit a marked capacity to interact with GAG, and in particular with HS. In this work we characterized CXCL12γ tissue expression and its capacity to promote CXCR4-dependent cell activation. Moreover, we characterized the interaction of CXCL12γ with GAG and the in vivo functions of this novel isoform. Our findings indicate that CXCL12γ, thanks to its sustained and high affinity for HS, exhibits an unprecedented chemokine activity that make it paradigmatic among haptotactic proteins, which regulate directional cell migration and promote tissue homing of many cell types.

Methods

Chemokine Synthesis and Monoclonal Antibodies

Chemically synthesized chemokines were generated by the Merrifield solid-phase method as described²⁴. The monoclonal antibody (mAb) 6E9 (IgG1κ) directed against the wild type CXCL12γ protein (thereafter called γ-wt for the recombinant and chemically synthesized proteins) was generated by immunizing BALB/c mice with a linear peptide containing the last 30 amino-acids of the γ-wt mature isoform, as previously described²⁴. The mAb clone K15C was generated against an amino-terminal peptide of CXCL12α (thereafter called α-wt for the recombinant and chemically synthesized proteins) shared by all the CXCL12 proteins.

Heparin Affinity Chromatography. Surface Plasmon Resonance (SPR)-Based Binding Assay (Biacore System)

Heparin affinity chromatography of chemokines was performed as previously described²⁵ on a 1-m1 Hitrap heparin column and submitted to gradient elution from 0.15 to 1 M NaCl in 20 mM Na2HPO4/NaH2PO4. For SPR experiments, size defined heparin (HP) (6 kDa) was biotinylated at its reducing end and immobilized on a Biacore sensorchip as described²⁵. For binding assays, 250 μl of chemokine was injected at a flow rate of 50 μl/min across control and HP surfaces, after which the formed complexes were washed with running buffer for 5 min. The sensorchip surface was regenerated with a 3 minutes pulse of 2 M NaCl. Control sensorgrams were subtracted on line from HP sensorgrams. Equilibrium data were extracted from the sensorgrams at the end of each injection and K_{d} were calculated using the Scatchard representation.

Cloning of Cxcl12 Isoforms.

Cxcl12α, β and γ cDNA sequences were isolated from a BALB/c mouse brain sample using the forward and reverse primer pairs 5′ tgcccttcagattgttgcac3′(SEQ ID NO: 2) and 5′ gctaactggttagggtaatac3′ (SEQ ID NO: 3) for Cxcl12α, 5′ gctttaaacaagaggctcaag3′ (SEQ ID NO: 4) and 5′ cctcctgcctcagctcaaag3′ (SEQ ID NO: 5) for Cxcl12β, and 5′ tgcccttcagattgttgcac3′ (SEQ ID NO: 6) and 5′ gcgagttacaaagcgccagagcagagcgcactgcg3′ (SEQ ID NO: 7) for Cxdl12γ. First-strand cDNA was synthesized and amplified sequences were subcloned in a pcDNA3.1 expression vector or in a plasmid containing the Semliki Forest Virus (SFV) genome deleted for structural genes (pSFV-1). For ease of detection, the sequence coding for the bovine rhodopsin C9-tag (TETSQVAPA-SEQ ID NO: 8) was added in frame at the 3′ end of the open reading frames (ORFs) of the Cxcl12α- and Cxcl12γ-encoding constructs, giving rise to the α-wt C9 and γ-wt C9 proteins, respectively. Nucleotide substitution (serine-coding triplets) in the Cxcl12γ-C9 construct corresponding to K⁷⁸_{/K}⁸⁰ or _{K}⁸⁶_{/K}⁸⁸ gave origin to the γ-C9^{up} and γC9^{dw} proteins, respectively.

Expression of Cxcl12 Isoforms

Production of defective SFV particles and infections were performed as described²⁶. pcDNA3.1 constructs were transfected in HEK-293T cells by the calcium phosphate method. Culture supernatants from 18 hr-SFV infected or 48 hr-transfected cells were collected and cleared by centrifugation. For preparing cell lysates, cells were detached in PBS-EDTA, centrifuged and pellets were treated with lysis buffer (20 mM Tris, pH 7.5, 100 mM (NH₄₎₂ SO₄, 10% Glycerol, 1× protease inhibitor and 1% Triton X-100) and thereafter, cleared by centrifugation. In some experiments, cells were washed for 5 minutes at 4° C. with PBS or 1M NaCl solution prior to cell lysis and centrifuged before collecting wash fluids.

Semi-Quantitative RT-PCR

Tissues were obtained by dissection of BALB/c adult mice, aliquoted and conserved in liquid N₂. Total RNA were obtained by using the Trizol reagent (Roche, Basel, Switzerland) and after phenol-chloroform purification, isopropanol precipitation and quantization, cDNA was synthesized using 1 μg of total RNA. The PCR reaction was carried out using the forward primer 5′ cccttcagattgttgcac3′ (SEQ ID NO: 9), common for all isoforms, and the isoform specific reverse primers 5′ taactggttagggtaatac3′ (SEQ ID NO: 10), 5′ tgagcctcttgtttaaagc3′ (SEQ ID NO: 11), and 5′ agttacaaagcgccagagcagagcgcactgcg3′ (SEQ ID NO: 12) for Cxdl12α, Cxcl12β and Cxcl12γ, respectively.

Immunostaining

Cells expressing C9-tagged chemokines were washed in PBS containing 0.5% BSA, left untreated or permeabilised with PBS 0.5% BSA, 0.05% saponin buffer for 30 min at 4° C., immunolabelled with the anti-C9-tag 1D4 mAb (Millipore, Billerica, USA) and finally revealed with a PE-conjugated secondary antibody. Confocal microscopy detection of CXCL12 chemokines was performed on brefeldin A-treated, fixed cells after saponin permeabilisation in a direct Microscope Widefield ApoTome Coolsnap. CXCR4 detection was performed with the PE-conjugated anti-human CD184 (clone 12G5; BD Biosciences, San Jose, Calif.).

Binding of CXCL12 chemokines to HMVEC, CHO-K1 or GAG-deficient CHO cells was assessed by incubation with the different chemically-synthesized chemokines followed by extensive washes. Labeling was performed with the pan anti-CXCL12 mAb clone K15C followed by a PE-conjugated secondary antibody. Cells were analysed by flow cytometry in a FacsCalibur (BD Biosciences). For immunohistochemistry experiments, paraffin-embedded, mouse tissue sections were incubated overnight at 4° C. with primary mAb K15C or the anti-γwt mAb (clone 6E9). Sections were washed and incubated with an anti-IgG mouse alkaline phosphatase ( 1/200) for 1.5 hr. Immunostaining was revealed using NBT/BCIP as a substrate. Human tissue immunostaining was revealed with an anti-IgG mouse biotinylated antibody and avidin-peroxidase system. Quantification of chemokines was carried out using the DuoSet ELISA Development kit for mouse CXCL12 (R&D Systems, MN, USA).

Functional Assays

For G protein-coupling assays, preparation of crude membrane fractions and [³⁵S]GTγS binding were performed as described²⁷. Migration of the lymphoblastoid cell line A3.01 or human primary CD4+ cells isolated from healthy donors as described²⁸ in response to CXCL12, was evaluated using a transwell system as described²⁸.

Intraperitoneal Recruitment Assay

Two-month-old female BALB/c mice received intraperitoneal injection of 300 μl of a 33 nM solution of the corresponding chemokine in PBS, using PBS alone as a control. Total peritoneal cells were recovered by washing the peritoneum with 20 ml of steril PBS. Total number of cells per mouse was determined by trypan blue exclusion and they were phenotyped by flow cytometry analysis using the mAbs FITC-rat anti-mouse Gr-1, FITC-hamster anti-mouse CD3, PE-rat anti-mouse CD11b or APC-rat anti-mouse CD19 (all from BD Biosciences). Cell influx in CXCL12-treated mice was calculated as the x-fold increase over negative control (PBS-treated mice).

Angiogenesis assay Mouse subcutaneous Matrigel® implants (BD Biosciences) were used as described²⁹. Briefly, 500 μl of Matrigel® containing 10 nM concentration of chemokines were subcutaneously injected in the back skin of female 2-mo-old BALB/c mice. The major component of Matrigel® is laminin, followed by collagen IV, heparan sulfate proteoglycans, and entactin³⁰. After 10 days, skin containing Matrigel® plugs were excised. Frozen sections were fixed in 4% paraformaldehyde and analysed by haematoxylin-eosin staining or immunofluorescent labelling. Phenotyping of endothelial cells was carried out by immunofluorescent labelling with an anti-CD31/PECAM-1 (Platelet Endothelial Cell Adhesion Molecule) mAb (Santa Cruz, Calif., USA). Quantitative data were obtained by counting the number of cells (DAPI positive nuclei) per Matrigel® area in digitalised images.

Results

Tissue Distribution of Cxcl12γ Products

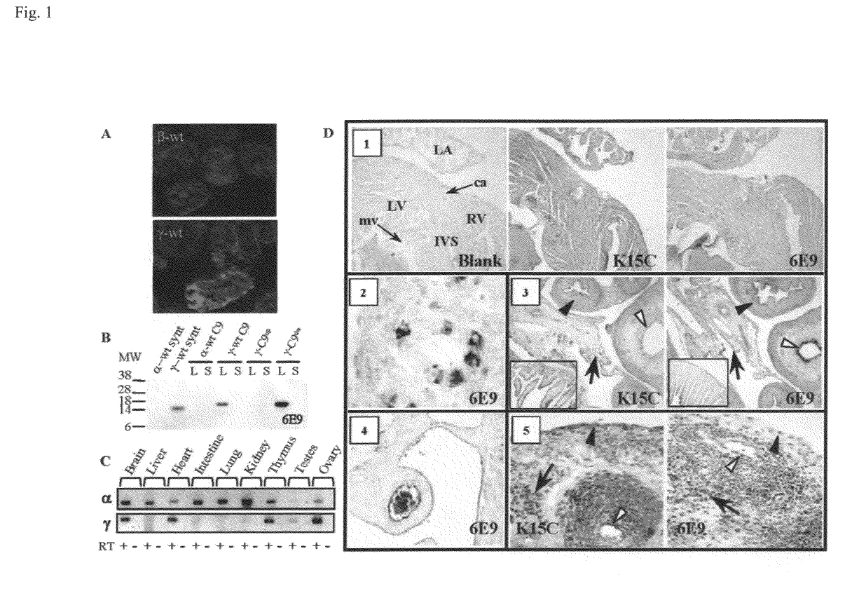

The Cxcl12γ isoform cDNA was obtained from BALB/c mouse brain mRNA. The isolated cDNA nucleotide sequence was identical to the previously reported murine Cxcl12γ isoform (NM_{—}001012477 NCBI acc. no.) that encodes the γ-wt protein (NP_{—}001012495 NCBI acc. no.). The expression of the γ-wt mRNA and protein in both embryo and adult mouse tissues and in human adult tissues, was investigated by RT-PCR and by immunohistochemistry using a novel mAb (6E9) that recognizes selectively a γ-wt C-ter epitope encompassing the sequence K78E79K80 (FIGS. 1A and 1B). The γ-wt protein expression was compared to these of other isoforms detected by the well characterized K15C mAb, which recognizes an amino-terminal-encoded epitope shared by all the CXCL12 isoforms^{31,32}.

In adult mice, the Cxcl12γ mRNA was poorly expressed in renal, bladder and intestinal epithelia, contrasting with the abundant expression of Cxcl12α mRNA (FIG. 1C). Regarding the protein (FIG. 1D), γ-wt was undetectable in bladder muscular and mucosa layers, while in the intestinal tract, a faint and discontinuous immunostaining was restricted to the mucosa and excludes the muscular layer (FIG. 1D, panel 3). Cxcl12γ mRNA was abundant in brain, heart (FIG. 1C) and BM, where it was expressed as a predominant isoform akin to Cxcl12α as quantified by Real-Time PCR (data not shown). γ-wt protein was detected in cardiac muscle, valves and large vessels (FIG. 1D, panel 1). In lungs and trachea, Cxcl12γ mRNA expression was abundant during organogenesis and barely detected in adult lung (FIG. 1C). Interestingly, a detailed analysis of γ-wt expression in mouse embryos showed that while the protein was virtually absent from trachea and large bronchia, it accumulated in the bronchioli (FIG. 1D, panel 2). The γ-wt protein was consistently detected in mesothelial tissues such as peritoneum (FIG. 1D, panel 3) and pleura (data not shown). Of note, γ-wt was detected in endothelia of large and small vessels both in human and mouse (FIG. 1D, panel 4 and 5), and in fibroblasts either of human skin (data not shown) or synovial inflammatory tissue (rheumatoid arthritis; FIG. 1D, panel 5).

γ-wt Binds to Immobilized and Cell Surface HS with High Affinity

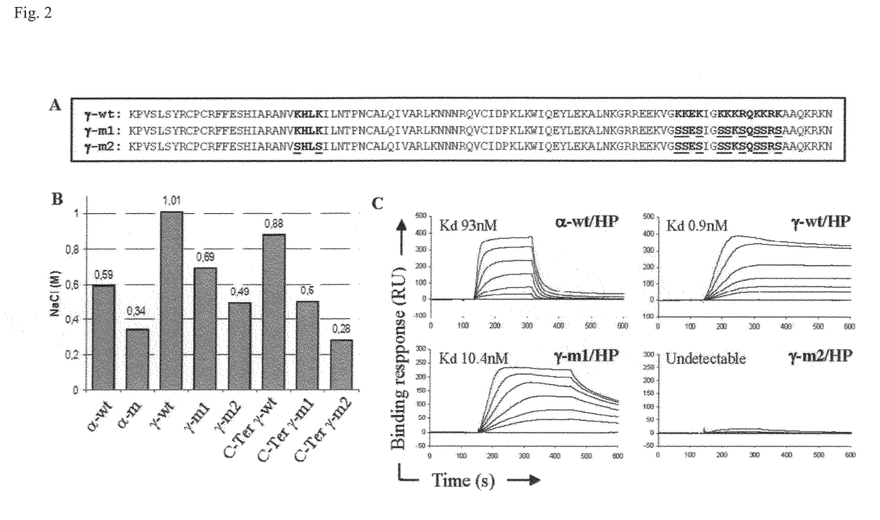

Previously it has been shown that α-wt binds with high affinity to HS²⁴ both in vitro and in intact cells through specific interaction with the canonical HS-binding motif (K24H25L26K27) located in the core of the protein shared by all the CXCL12 isoforms. Mutation of this motif (K24S/K27S) fully prevents binding to HS without affecting neither the overall structure nor the capacity of the mutant chemokine (α-m) to bind and activate CXCR4²⁴. The specific C-ter domain of the γ-wt isoform presents a marked basic character, with a 60% of the residues being positively charged and clustered in 4 overlapped HS-binding sites. This prompted us to investigate the γ-wt/GAG interactions. Analysis performed with chemically synthesized chemokines, showed that γ-wt isoform required 1.01 M NaCl to be eluted from a HP-affinity column (FIG. 2B) as compared to 0.59 M required for elution of αwt. Chemically synthesized γ-wt C-ter peptide encompassing amino-acids 69 to 98 required 0.88 M NaCl to be eluted, indicating that this domain interacts with HP per se with high affinity and might contributes to the strong interaction with HP displayed by γ-wt. In good agreement, neutralization of positively charged amino-acids by mutation of the C-ter BBXB motifs either in the γ-wt (γ-m1, FIG. 2A) or the isolated C-ter peptide, reduced drastically the ionic force (0.69 and 0.5 M NaCl, respectively, FIG. 2B) required for their elution from the HP-affinity column.

Surface Plasmon Resonance (SPR, Biacore system, FIG. 2C) experiments confirmed that γ-wt interacts with HP with unprecedented high affinity (K_{d}=0.9 nM). Furthermore they showed that the interaction with the oligosaccharide is severely impaired in the mutant γ-m1 (K_{d}=14 nM), thus proving the important contribution of the C-ter domain BBXB sites to the binding on HP. Both HP-affinity chromatography and SPR experiments (FIGS. 2B and 2C) proved that the γ-m2 mutant (FIG. 2A), which lacks functional BBXB motifs, is virtually devoid of the capacity to interact with HP.

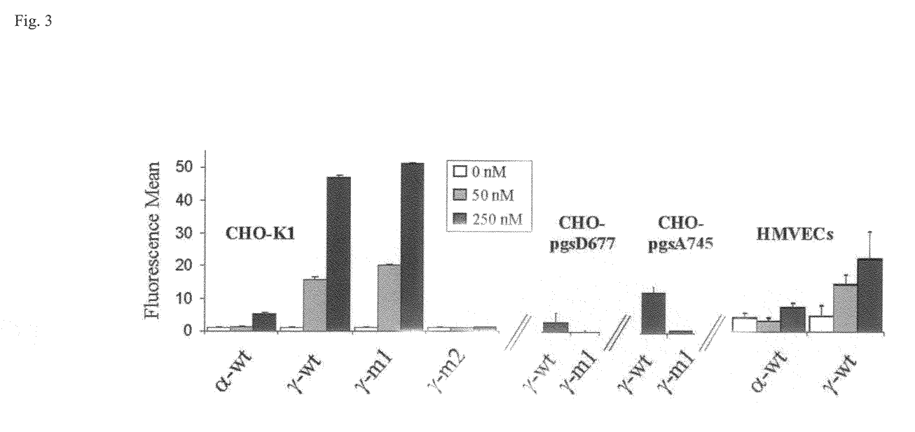

Recognition of CXCL12 proteins by the K15C mAb is not masked by their interaction with GAG²⁰. Using this mAb, we observed that the adsorption on CHO-K1 CXCR4 negative cells is greatly increased for γ-wt as compared to α-wt (FIG. 3). The γ-m1 mutant protein retains a substantial capacity to bind CHO-K1 parental cells, whereas the γ-m2 is virtually devoid of any binding capacity on cell surface. In CHO-pgsD677 cells, derived from CHO-K1 cells, which lack both N-acetylglucosaminyltransferase and glucuronyltransferase activities and are deficient for HS synthesis, the binding of γ-m1 became undetectable, whereas a residual signal was still detectable for γ-wt. A similar phenomenon was observed in CHOpgsA745 cells, which lack any GAG synthesis due to a xylose-transferase mutation. The residual binding observed at very high concentrations of the chemokine, in the absence of any synthesized GAG, can be accounted for by the abundant sulphate glycosphingolipids (sulphatides) that have been previously shown to interact at high concentrations with α-wt³³.

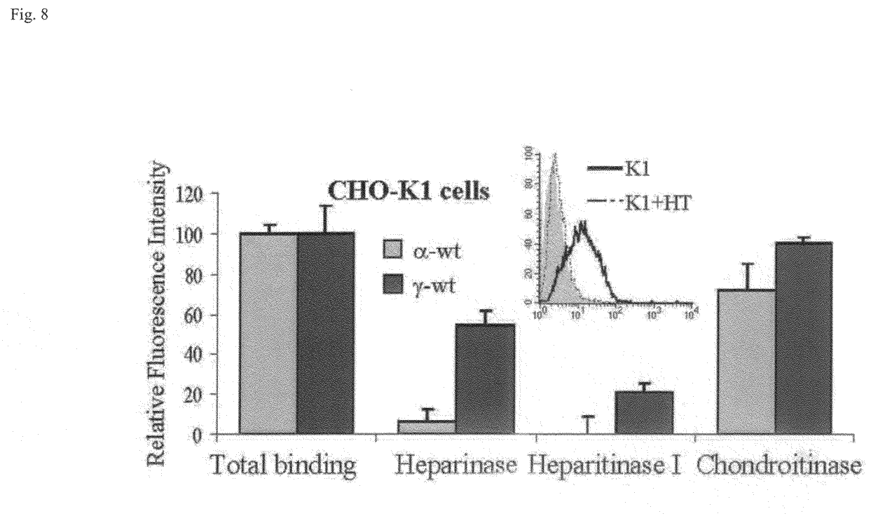

The enzymatic degradation of HS in CHO-K1 cells either by heparinase or heparitinase I, an enzyme with increased catalytic stability, confirmed the apparent selectiveness of the HS/γ-wt interaction at the cell surface, whereas degradation of chondroitin sulfates had no effect (FIG. 8). Of note, and of particular biological relevance, we found that γ-wt also binds onto primary, human-microvascular endothelial cells (HMVEC) with the highest efficiency as compared to α-wt (FIG. 3).

Neosynthesized γ-wt Shows an Unusual Pattern of Cell Secretion and Accumulation

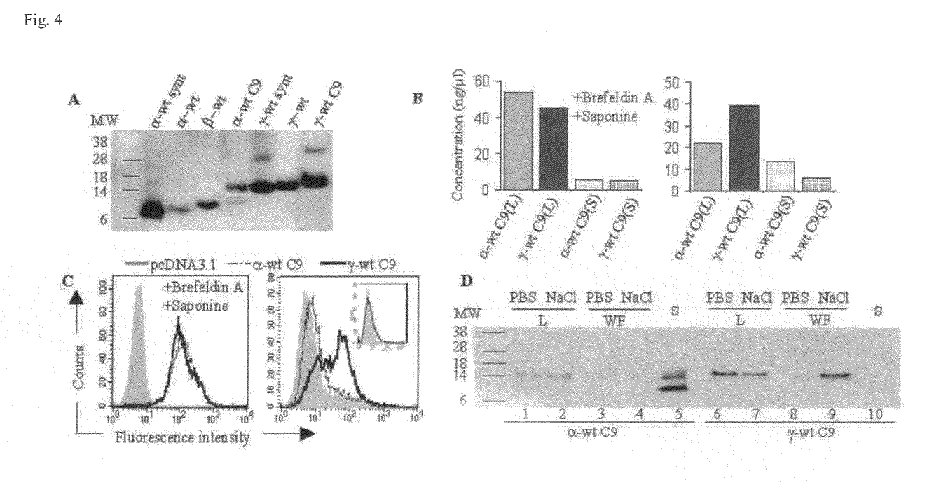

We observed that the CXCL12γ C9-tagged (γ-wt C9) protein was hardly detectable by western blot analysis in the cell culture supernatants of expressing cells (FIGS. 1B and 4D). This finding, prompted us to investigate the fate of this protein and to compare it to α-wt C9 engineered and expressed under identical experimental conditions (FIG. 4A). Quantification in an ELISA assay of either cell-associated (cell lysate) or free chemokines (supernatant) showed that similar amounts of γ-wt C9 and α-wt C9 were produced from expressing cells treated with brefeldin A (FIG. 4B, left panel). Moreover, we observed that a larger fraction of γ-wt C9 remained associated to cells as compared to α-wt C9 (FIG. 4B, right panel). To further investigate the distribution of the chemokine fraction associated to cells, we performed labeling of α-wt C9- and γ-wt C9-expressing cells using the anti-C9 1D4 mAb (FIG. 4C). Interestingly, γ-wt C9 markedly amassed at the cell surface in contrast to α-wt C9 that was barely detected at this level (FIG. 4C, right panel), while we confirmed that in the presence of brefeldin A, similar amounts of each chemokine accumulated in intracellular stores (FIG. 4C, left panel). Enzymatic exposure to heparitinase I reduced the intensity of the signal for both chemokines (data not shown), which is in full agreement with the previous SPR data showing that γ-wt binds with exceptionally high affinity to on-chip immobilized HS. These findings lead us to postulate that given the cationic nature of the C-ter of γ-wt and its high affinity for GAG, electrostatic forces enable this chemokine to bind tightly to negatively charged structures at the cell surface. This assumption was tested by exposing shortly SFV-infected BHK cells expressing either γ-wt C9 or α-wt C9 to isotonic PBS or 1M NaCl solution, in order to disrupt the electrostatic interactions and eventually promote release of the chemokine into the fluid (FIG. 4D). While α-wt C9-expressing cells did not release detectable amounts of this isoform in the wash fluid (FIG. 4D lanes 3 and 4), a significant amount of γ-wt C9 was released upon short exposure of cells to 1M NaCl solution (FIG. 4D lane 9). These findings were reproduced in other cell types (HEK 293T) and with different expression vectors (pcDNA3.1).

γ-wt Shows Reduced Agonist Potency on CXCR4 Activation as Compared to α-wt.

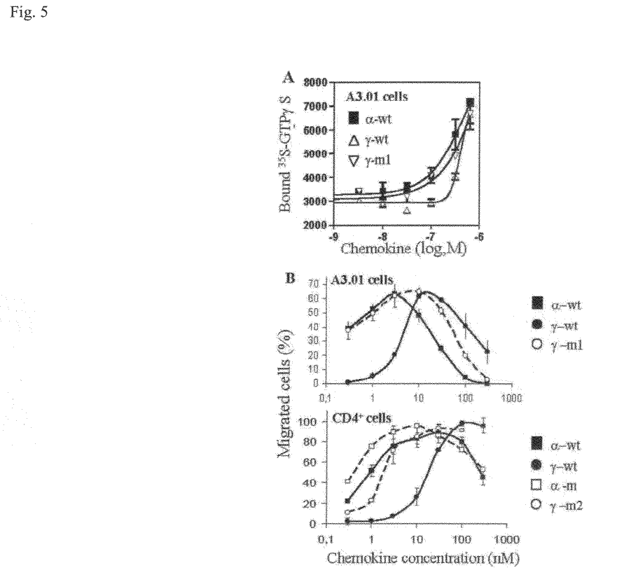

The pharmacological properties of γ-wt regarding its interaction with CXCR4 were investigated on transformed A3.01 T cells and primary CD4+ T lymphocytes (FIG. 5). Both lymphoid cell types lack detectable levels of HS as assessed by immunostaining with the specific 10E4 anti-HS mAb (data not shown) and permits the strict analysis of CXCL12/CXCR4 interaction per se. The capacity of γ-wt to set in motion CXCR4-dependent activation cell pathways was first assessed by measuring the amount of the non-hydrolysable [S³⁵]-GTPγ associated to activated Gα subunits, the earliest cell-signal event induced by GPCR agonists. We show that γ-wt is less potent than α-wt to activate CXCR4 (FIG. 5A), which is in full agreement with the reduced affinity (one order of magnitude) shown by γ-wt for CXCR4 as compared to α-wt (C. L. and R. S., manuscript submitted September 2007). When concentration of the γ-wt was raised and the occupancy of CXCR4 was enhanced, CXCR4 activation increased and reached the level achieved by α-wt. This indicates that, although they differ by their potency, the pharmacological efficiency of α-wt and γ-wt are equivalent. Of note, γ-m1 that binds CXCR4 as efficiently as α-wt (C. L. and R. S., manuscript submitted September 2007), proved to be consistently a better agonist than γ-wt.

We next investigated the capacity of γ-wt to promote orientated lymphocyte migration, the hallmark of chemokine-promoted cell responses. Addition of γ-wt to A3.01 cells or primary, CD4+ T lymphocytes resulted in a bell-shaped dose response characteristic of chemokine stimulation and confirmed the reduced potency of γ-wt as compared to α-wt (FIG. 5B). Invalidation of the C-ter BBXB motifs in γ-wt (γ-m1 and γ-m2) restores the potency of the chemokine to the levels showed by α-wt and α-m, two chemokines that have been

previously shown to bind and activate CXCR4 similarly²⁴, and prove that the mutations introduced in the C-ter domain do not affect the overall structure of the chemokine. Addition of the specific CXCR4 antagonist AMD3100 resulted in the blockade of G-protein coupling and cell migration thus proving the specificity of CXCR4/γ-wt interactions (data not shown).

In Vivo Biological Activity of γ-wt

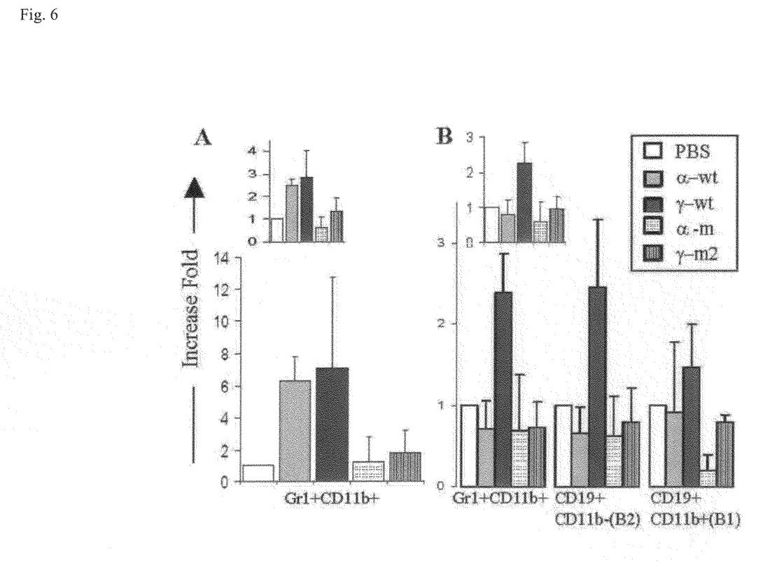

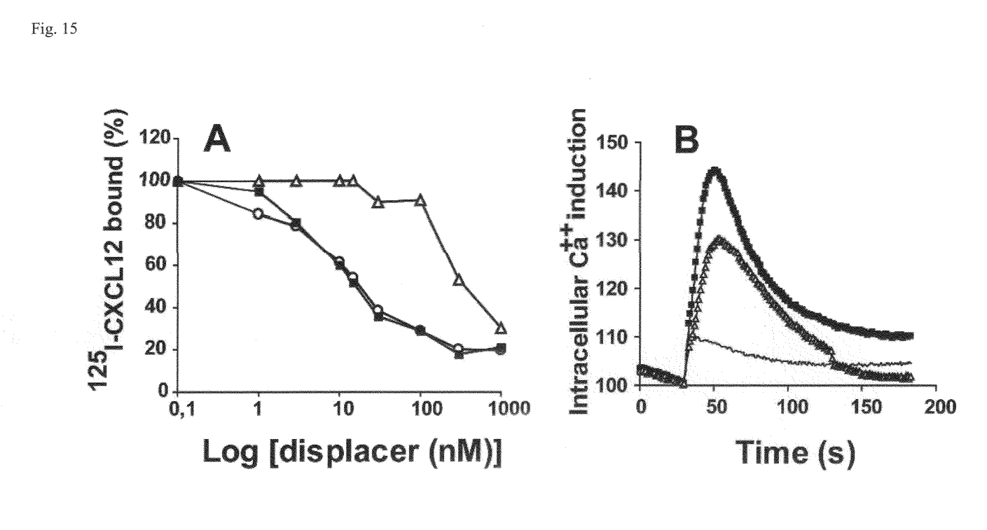

The singular structural and functional features that distinguish γ-wt from α-wt prompted us to compare their respective capacities to promote haptotactic attraction of cells in vivo. To this purpose, we first evaluated the migration of leukocytes into the peritoneal cavity of BALB/c mice at 6 hr (FIG. 6A) and 15 hr (FIG. 6B) following administration of γ-wt or α-wt. After 6 hr of treatment, both α-wt and γ-wt induced an equivalent increase of the absolute number of cells (2.5±0.3 and 2.9±1.1 fold increase, for α-wt and γ-wt respectively) as compared to control animals injected with PBS. At this time, only neutrophils (Gr-1+ CD11b+ CD19−) were significantly attracted either by α-wt or γ-wt (roughly 6-fold increase). The situation was radically different 15 hr post-injection, as solely γ-wt promoted a sustained accumulation of neutrophils (2.4±0.5) and also B lymphocytes, including B1 (CD19+ CD11b−) and B2 (CD19+ CD11b+) cell subsets (1.47±0.5 and 2.5±0.7 fold increase, respectively). In sharp contrast, both α-m and γ-m2 that totally lack HS-binding activity, failed to attract leukocytes at any time point.

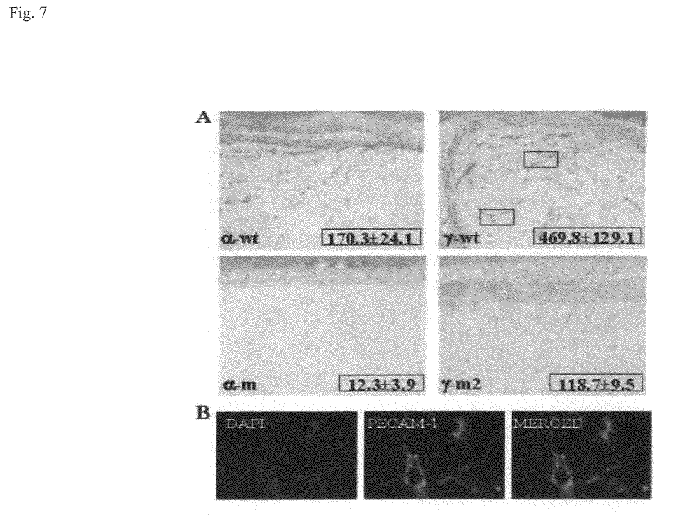

CXCL12α owns the capacity to promote de novo formation of vessels, a property related to the ability of this chemokine to regulate both the traffic and survival of stem and progenitor cells^{16,34}. On this basis, we compared the ability of γ-wt and α-wt to attract endothelial progenitors and initiate the angiogenic process. To this purpose, Matrigel® plugs loaded with equimolar amounts either of γ-wt or α-wt were implanted subcutaneously in BALB/c mice. Whereas virtually no infiltrating cells were detectable in control PBS Matrigel® plugs (data not shown), γ-wt induced a more robust response (3-fold increase, FIG. 7A) than α-wt regarding the total number of cells attracted. Vessel-like cellular tubes within Matrigel® implants were particularly abundant in γ-wt-loaded implants. These vessel-like structures were mainly composed of endothelial cells expressing PECAM-1/CD31 (FIG. 7B), a molecule that defines endothelial progenitor cells and participates in adhesive and/or cell-signaling phenomena required for the motility of endothelial cells and/or their subsequent organization into vascular tubes. Of note, both α-m and γ-m2 display a reduced capacity to promote cell infiltration and angiogenesis in Matrigel® implants, demonstrating the importance of GAG binding for this process.

Discussion

The expression of Cxcl12γ has been reported to be expressed preferentially in the central nervous system of adult rats and it is supposed to undergo inverse regulation as compared to the β isoform²². Cxcl12γ transcripts are detected broadly in human tissues while in mice its expression has been observed in the brain³⁵. The expression pattern of γ-wt during organogenesis suggests the participation of this isoform in the development of cardiovascular and immune system, both regulated by Cxcl12. The apparent exclusion of both Cxdl12γ mRNA and protein from several epithelia suggests that the expression of this isoform is tightly regulated by a RNA-splicing regulatory mechanism. Remarkably, CXCL12γ seems to be expressed in anatomical sites such as small vessels and lower respiratory tract, where it could be involved in the diapedesis of inflammatory leukocytes and other cells from hematopoietic origin. Its expression in embryo and its enhanced capacity to form haptotactic gradients could be the mechanism by which, discrete cell precursors are guided into their final localization during organogenesis.

The tight array of BBXB motifs in the CXCL12γ C-ter domain that distinguish this protein from other CXCL12 isoforms, is unprecedented among HS-binding proteins. The Cter domain has on its own a marked affinity for heparin that decreases dramatically when HS-binding motifs are mutated and invalidated. This observation is in keeping with our results issued from a Nuclear Magnetic Resonance analysis of the soluble form of this chemokine (C. L. and R. S., manuscript submitted September 2007), which revealed that the C-ter peptide is unfolded and could offer an accessible, highly cationic surface for the interaction with GAG for molecular recognition. Our interpretation of SPR findings is that, the high affinity for the oligosaccharide displayed by γ-wt largely relies in the low k_{off} of the HP-γ-wt complexes which has been estimated to be 0.0019 M⁻¹s⁻¹, contrasting with the rapid dissociation from HP observed for α-wt (k_{off} 0.111 M⁻¹s⁻¹) (C. L. and R. S., manuscript submitted September 2007). This is well exemplified by the SPR profile obtained with the mutant γ-m1. This mutant dissociates more rapidly from HP and shows a marked, reduced interaction with HP as compared to the wild type counterpart. However, it retains a substantial affinity for HP that might result from the stabilization of the complex through the collaboration between the conserved BBXB motif in the core of the chemokine and the remaining positive charges in the yet highly cationic C-ter domain. Collectively, these data underline the important contribution of the C-ter BBXB motifs to the formation of high-affinity and stable γ-wt/HS complexes.

Additional data prove the central role of the highly-cationic C-ter domain in providing CXCL12γ with an exceptional capacity to bind heparan sulfates. Indeed, when, the protein BBXB motif shared by all the isoforms is mutated leaving intact the sequence of the last 30aa that constitute the C-ter domain of CXCL12γ, the resultant functional CXCL12 mutant maintains a preserved capacity to interact with heparan sulfates either immobilized on sensorchips or expressed on cell surfaces at an extent roughly comparable to this of the wild type protein. The CXCR4 agonist capacity of the mutant CXCL12 is equivalent to this of the wild type CXCL12 γ. These findings confirm and extend our conclusions regarding the central role played by the C-ter domain on the functional characteristics of CXCL12γ and reinforces our assumption that this domain encodes, per se, a functional and transposable domain that could in cis confers to heterologous proteins the capacity to interact with HS featured by CXCL12γ.

The chemokine-binding experiments carried out in intact cells proved the specificity and high affinity of γ-wt for cellular HS structures, thus validating the biological relevance of the in vitro analysis. The astounding strong interaction of γ-wt with cell GAG was also observed in an alternative assay. Indeed, our findings prove that γ-wt is massively adsorbed at the cell surface following secretion. The simplest explanation for this phenomenon is that the secreted γ-wt could be rapidly trapped on cell-surface HS structures. Alternatively, the high affinity of γ-wt for HS might result in the formation of an intracellular complex before being expressed at the cell surface, a phenomenon previously described for the Fibroblast Growth Factor-2³⁶. On view of the low dissociation rate of HS-γ-wt complexes it can be speculated that the secreted, free form of the chemokine hardly would reach the equilibrium of interaction with immobilized HS and that under physiological conditions, the binding of natural CXCL12γ to extracellular HS structures is tight and long-lasting.

Using lymphoid T cells, we demonstrate that γ-wt signals through CXCR4 with diminished agonist potency that can be accounted for by the diminished affinity that this chemokine shows for CXCR4 as compared to α-wt. It can be hypothesized that, either the electrostatic interactions of the highly cationic C-ter domain with the negatively charged N-ter domain and extracellular loops of CXCR4³⁷, or the steric hindrance promoted by the bulky basic residues in the γ-wt C-ter domain, impair the specific interaction with CXCR4 and therefore reduce the agonist potency of γ-wt. Consistent with this data and hypothesis, we did not observed either in transformed or primary CD4 T lymphocytes, the supposed supremacy of γ-wt over α-wt regarding the inhibition of CXCR4-tropic HIV infection³⁸ (data not shown).

Importantly, neutralisation of positive charges in the BBXB motif of γ-wt reverts CXCR4 activation to the levels observed either for α-wt or α-m, two proteins which have been demonstrated previously not to differ on their overall structure and to bind and activate CXCR4 with akin efficiency²⁴. Moreover, the γ-m1 chemokine shows roughly the same affinity for CXCR4 as α-wt (C. L. and R. S., manuscript submitted September 2007). Collectively, these findings conclusively identify the charged C-ter domain as responsible for the distinctive structural and cell-signaling properties showed by γ-wt.

The demonstration of in vivo consequences of chemokine/GAG interactions have been hampered by conformational changes consecutive to the mutagenesis of BBXB consensus sites that leads frequently to an overall reduced affinity of the chemokine for the corresponding receptor. The naturally occurring CXCL12γ protein is free of this bias and offers an unprecedented opportunity to ascertain the importance of chemokine/GAG complexing in the regulation of in vivo cell migration in adult life.

The capacity to promote leukocyte attraction in the peritoneum by endogenous CXCL12α has been proved previously³⁹. Similarly it has been demonstrated that, the formation of vessels under physiological and pathological conditions¹⁷ is induced by CXCL12 and is related to the regulation of the traffic and survival of CD34+ progenitor cells. The in vivo findings shown in this work show the superior biological efficiency of γ-wt over α-wt. The animal models used in this work are pertinent to in vivo situations as we demonstrate that both chemokines are expressed in mesothelial cells from the coelomic cavities and are detected in, and bind to, endothelial cells. We show that the preserved HS-binding capacity of the chemokine is critical for the induction of robust in vivo effects since both HS-binding disabled γ-m2 and α-m mutants are virtually inactive.

CXCL12γ is apparently constitutively expressed in a number of organs and tissues and it can be speculated that its long-lasting HS binding facilitates the constitution of a chemokine reservoir. The exposed C-ter domain of this isoform encodes several consensus sites for serine proteases. Thus, it is conceivable that in response to pathogens and tissue damage, a free, functional chemokine could be released either from cell- or matrix-binding. However, when in complex with HS, the oligosaccharide might restrain the access of proteases to the C-ter domain, a situation previously reported for the CXCL12 N-ter and its inactivation by CD26²⁰. As a consequence, γ-wt could associate stably with HS in vivo and therefore promote a sustained haptotactic attraction of cells. This assumption is strongly supported by the fact that γ-m2 is devoid of any significant in vivo activity, in spite of the increased agonist potency of this mutant and its enhanced ability to promote leukocyte chemotaxis in vitro.

The conserved structure and differential expression of CXCL12γ herald the important role that it might play both during development and in adult life. Its localization at anatomical sites where leukocyte diapedesis occurs and pathogen host defence are initiated, suggests that this chemokine is key in the fine-tuning of immune responses. We conclude that CXCL12γ represents the paradigm of haptotactic proteins that critically promote the directional migration and tissue homing of cells and regulate important homeostatic and physiopathological functions.

### REFERENCES FOR EXAMPLE 1

1. Science. 1993; 261:600-603.
2 Shirozu M, Nakano T, Inazawa J, et al. Structure and chromosomal localization of the human stromal cell-derived factor 1 (SDF1) gene. Genomics. 1995; 28:495-500.
3 Ieul C C, Farzan M, Choe H, et al. The lymphocyte chemoattractant SDF-1 is a ligand for LESTR/fusin and blocks HIV-1 entry. Nature. 1996; 382:829-833.
4 Nagasawa T, Tachibana K, Kishimoto T. A novel CXC chemokine PBSF/SDF-1 and its receptor CXCR4: their functions in development, hematopoiesis and HIV infection. Semin Immunol. 1998; 10:179-185.
5 Zou Y R, Kottmann A H, Kuroda M, Taniuchi I, Littman D R. Function of the chemokine receptor CXCR4 in haematopoiesis and in cerebellar development. Nature. 1998; 393:595-599.
6 Klein R S, Rubin J B, Gibson H D, et al. SDF-1 alpha induces chemotaxis and enhances Sonic hedgehog-induced proliferation of cerebellar granule cells. Development. 2001; 128:1971-1981.
7 Ma Q, Jones D, Borghesani P R, et al. Impaired B-lymphopoiesis, myelopoiesis, and derailed cerebellar neuron migration in CXCR4- and SDF-1-deficient mice. Proc Natl Acad Sci USA. 1998; 95:9448-9453.
8 Ara T, Nakamura Y, Egawa T, et al. Impaired colonization of the gonads by primordial germ cells in mice lacking a chemokine, stromal cell-derived factor-1 (SDF-1). Proc Natl Acad Sci USA. 2003; 100:5319-5323.
9 Shamri R, Grabovsky V, Gauguet J M, et al. Lymphocyte arrest requires instantaneous induction of an extended LFA-1 conformation mediated by endothelium-bound chemokines. Nat. Immunol. 2005; 6:497-506.
10 Kantele J M, Kurk S, Jutila M A. Effects of continuous exposure to stromal cell-derived factor-1 alpha on T cell rolling and tight adhesion to monolayers of activated endothelial cells. J. Immunol. 2000; 164:5035-5040.
11 Grabovsky V, Feigelson S, Chen C, et al. Subsecond induction of alpha4 integrin clustering by immobilized chemokines stimulates leukocyte tethering and rolling on endothelial vascular cell adhesion molecule 1 under flow conditions. J Exp Med. 2000; 192:495-506.
12 Campbell J J, Hedrick J, Zlotnik A, Siani M A, Thompson D A, Butcher E C. Chemokines and the arrest of lymphocytes rolling under flow conditions. Science. 1998; 279:381-384.
13 Aiuti A, Webb I J, Bleul C, Springer T, Gutierrez-Ramos J C. The chemokine SDF-1 is a chemoattractant for human CD34+ hematopoietic progenitor cells and provides a new mechanism to explain the mobilization of CD34+ progenitors to peripheral blood. J Exp Med. 1997; 185:111-120.
14 Nanki T, Hayashida K, El-Gabalawy H S, et al. Stromal cell-derived factor-1-CXC chemokine receptor 4 interactions play a central role in CD4+ T cell accumulation in rheumatoid arthritis synovium. J. Immunol. 2000; 165:6590-6598.
15 Gallagher K A, Liu Z J, Xiao M, et al. Diabetic impairments in NO-mediated endothelial progenitor cell mobilization and homing are reversed by hyperoxia and SDF-1 alpha. J Clin Invest. 2007; 117:1249-1259.
16 Ceradini D J, Kulkarni A R, Callaghan M J, et al. Progenitor cell trafficking is regulated by hypoxic gradients through HIF-1 induction of SDF-1. Nat. Med. 2004; 10:858-864.
17 Burger J A, Kipps T J. CXCR4: a key receptor in the crosstalk between tumor cells and their microenvironment. Blood. 2006; 107:1761-1767.
18 Esko J D, Selleck S B. Order out of chaos: assembly of ligand binding sites in heparan sulfate. Annu Rev Biochem. 2002; 71:435-471.
19 Proudfoot A E, Handel T M, Johnson Z, et al. Glycosaminoglycan binding and oligomerization are essential for the in vivo activity of certain chemokines. Proc Natl Acad Sci USA. 2003; 100:1885-1890.
20 Sadir R, Imberty A, Baleux F, Lortat-Jacob H. Heparan sulfate/heparin oligosaccharides protect stromal cell-derived factor-1 (SDF-1)/CXCL12 against proteolysis induced by CD26/dipeptidyl peptidase IV. J Biol. Chem. 2004; 279:43854-43860.
21 Sweeney E A, Lortat-Jacob H, Priestley G V, Nakamoto B, Papayannopoulou T. Sulfated polysaccharides increase plasma levels of SDF-1 in monkeys and mice: involvement in mobilization of stem/progenitor cells. Blood. 2002; 99:44-51.
22 Gleichmann M, Gillen C, Czardybon M, et al. Cloning and characterization of SDF1 gamma, a novel SDF-1 chemokine transcript with developmentally regulated expression in the nervous system. Eur J. Neurosci. 2000; 12:1857-1866.
23 Yu L, Cecil J, Peng S B, et al. Identification and expression of novel isoforms of human stromal cell-derived factor 1. Gene. 2006; 374:174-179.
24 Amara A, Lorthioir O, Valenzuela A, et al. Stromal cell-derived factor-1 alpha associates with heparan sulfates through the first beta-strand of the chemokine. J Biol. Chem. 1999; 274:23916-23925.
25 Sadir R, Baleux F, Grosdidier A, Imberty A, Lortat-Jacob H. Characterization of the stromal cell-derived factor-1alpha-heparin complex. J Biol. Chem. 2001; 276:8288-8296.
26 Staropoli I, Chanel C, Girard M, Altmeyer R. Processing, stability, and receptor binding properties of oligomeric envelope glycoprotein from a primary HIV-1 isolate. J Biol. Chem. 2000; 275:35137-35145.
27 Lagane B, Ballet S, Planchenault T, et al. Mutation of the DRY motif reveals different structural requirements for the CC chemokine receptor 5-mediated signaling and receptor endocytosis. Mol. Pharmacol. 2005; 67:1966-1976.
28 Balabanian K, Lagane B, Pablos J L, et al. WHIM syndromes with different genetic anomalies are accounted for by impaired CXCR4 desensitization to CXCL12. Blood. 2005; 105:2449-2457.
29 Pablos J L, Santiago B, Galindo M, et al. Synoviocyte-derived CXCL12 is displayed on endothelium and induces angiogenesis in rheumatoid arthritis. J Immunol. 2003; 170:2147-2152.
30 Zimrin A B, Villeponteau B, Maciag T. Models of in vitro angiogenesis: endothelial cell differentiation on fibrin but not matrigel is transcriptionally dependent. Biochem Biophys Res Commun. 1995; 213:630-638.
31 Allen C D, Ansel K M, Low C, et al. Germinal center dark and light zone organization is mediated by CXCR4 and CXCR5. Nat. Immunol. 2004; 5:943-952.
32 Coulomb-L'Hermin A, Amara A, Schiff C, et al. Stromal cell-derived factor 1 (SDF-1) and antenatal human B cell lymphopoiesis: expression of SDF-1 by mesothelial cells and biliary ductal plate epithelial cells. Proc Natl Acad Sci USA. 1999; 96:8585-8590.
33 Sandhoff R, Grieshaber H, Djafarzadeh R, et al. Chemokines bind to sulfatides as revealed by surface plasmon resonance. Biochim Biophys Acta. 2005; 1687:52-63.
34 Orimo A, Gupta P B, Sgroi D C, et al. Stromal fibroblasts present in invasive human breast carcinomas promote tumor growth and angiogenesis through elevated SDF-1/CXCL12 secretion. Cell. 2005; 121:335-348.
35 Stumm R K, Rummel J, Junker V, et al. A dual role for the SDF-1/CXCR4 chemokine receptor system in adult brain: isoform-selective regulation of SDF-1 expression modulates CXCR4-dependent neuronal plasticity and cerebral leukocyte recruitment after focal ischemia. J. Neurosci. 2002; 22:5865-5878.
36 Nickel W. Unconventional secretion: an extracellular trap for export of fibroblast growth factor 2. J Cell Sci. 2007; 120:2295-2299.
37 Brelot A, Heveker N, Adema K, Hosie M J, Willett B, Alizon M. Effect of mutations in the second extracellular loop of CXCR4 on its utilization by human and feline immunodeficiency viruses. J. Virol. 1999; 73:2576-2586.
38 Altenburg J D, Broxmeyer H E, Jin Q, Cooper S, Basu S, Alkhatib G. A naturally occurring splice variant of CXCL12/stromal cell-derived factor 1 is a potent human immunodeficiency virus type 1 inhibitor with weak chemotaxis and cell survival activities. J. Virol. 2007; 81:8140-8148.
39 Foussat A, Balabanian K, Amara A, et al. Production of stromal cell-derived factor 1 by mesothelial cells and effects of this chemokine on peritoneal B lymphocytes. Eur J. Immunol. 2001; 31:350-359.

### Example 2

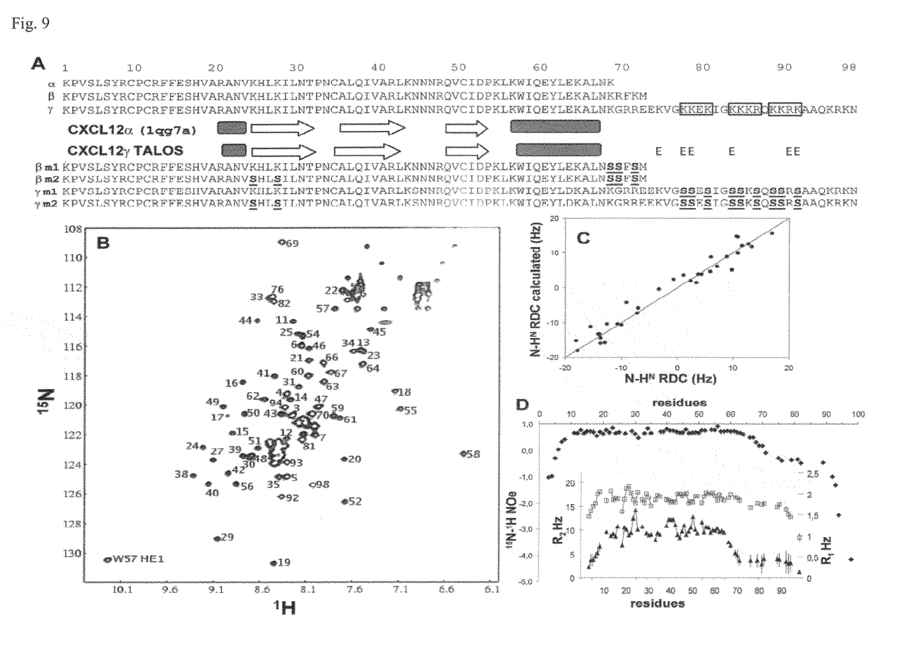

CXCL12, also known as SDF-1 (Stromal cell-Derived Factor-1), belongs to the growing family of chemokines, a group comprising some fifty low molecular weight proteins, best known to mediate leukocyte trafficking and activation [1]. CXCL12, initially identified from bone marrow stromal cells and characterized as a pre-B-cell stimulatory factor [2], is constitutively expressed within tissues during organogenesis and adult life [3,4]. This chemokine, highly conserved among mammalian species, is a key regulator of oriented cell migration and as such, orchestrates a very large array of functions both during development and adult life [5-9] but is also importantly involved in a number of pathogenic mechanisms [10,11]. These physiopathological effects, are mediated by the G-protein coupled receptor CXCR4, to which the chemokine binds and triggers cell signaling [6,12]. In addition to these physiological functions, CXCL12 is a potent inhibitor of the cellular entry of CXCR4-dependent human immunodeficiency virus [12]. Recently we have documented that CXCR7 (RDC-1), also binds to—and is activated by—CXCL12 [13] although the biological role played by this couple remains to be further characterized. From a structural view point, CXCL12 has a typical chemokine fold stabilized by two disulfide bonds: it consists of a poorly structured N-terminus of 10 residues, followed by a long loop, a 3₁₀ helix, a three stranded β-sheet and a C-terminal α-helix. Up to recently, two CXCL12 isoforms, arising from alternative splicing of a single gene [14] have been studied. The predominant α form encodes a 68 amino acid peptide while the β one contains four additional amino acids at the C terminus. Most functional data on CXCL12 were obtained from CXCL12α and β, while to date, three isoforms (α, β and γ) and up to six isoforms (α, β, γ, δ, ε and φ) of CXCL12 have been found in rodents [15] and human [16], respectively. All these isoforms share the same three first exons corresponding to the α isoform (residues 1 to 68), but differ in their fourth exon, which gives rise to a specific C-terminal domain for each of them. It has become clear that biological information required to run the chemokine systems is not only stored in the sequences of the proteins involved, but also in the structure of a class of polysaccharide called glycosaminoglycans (GAGs), in particular heparan sulfate (HS), to which most chemokines bind [17] primarily through ionic interactions. Anchored to various core proteins to form proteoglycans, these complex polysaccharides are ubiquitously found on the cell surface and within the extracellular matrix [18]. These molecules have a unique molecular design in which sulfated disaccharide units are clustered in specific domains of variable length and sulfation profile, providing the chain a large array of different protein binding sites [19]. HS are importantly implicated in the regulation of the proteins they bind, and have recently emerged as critical regulators of many events involving cell response to external stimuli. Current models suggested that HS enhances chemokine immobilization and forms haptotactic gradients of the protein along cell surfaces, hence providing directional cues for migrating cells [20], protects chemokines from enzymatic degradation [21], and promotes local high concentrations at the cell surface, facilitating receptor binding and downstream signaling (for review see [22]). In vivo data support the view that, within tissues, CXCL12 is sequestered by HS [23]. CXCL12α binding to HS critically involves amino acids K24 and K27, which together with R41 form the essential part of the HS-binding site [24] and are distinct from those required for binding to CXCR4. Given that the minor δ, ε and φ isoforms lack any recognizable HS-binding motif in their carboxy-termini, it can be hypothesized that like CXCL12α, the K24-K27-R41 epitope recapitulates their ability to interact with HS. The situation could be radically different for the novel CXCL12γ isoform. It is indeed characterized by a distinctive 30 amino acids long C-terminal peptide, remarkably conserved between rodents and human, which contains as much as 18 basic residues (B), 9 of which being clustered into three putative BBXB HS-binding domains (FIG. 9A). The existence of carboxyencoded HS-binding motifs suggests that this isoform could interact with enhanced affinity and/or different selectiveness with GAGs to accomplish specific functions. However, the structure/function relationships of this very peculiar CXCL12 isoform have not been explored. Here we show that CXCL12γ 1 to 68 domain adopts a structure closely related to the α isoform and has an unstructured C-terminal region. This domain reduces CXCR4 occupancy, but in contrast extends the type of GAG to which the chemokine binds. Moreover, it stabilizes the CXCL12γ/HS complex and, in cooperation with the K24-R41 epitope, provides the chemokine with the highest affinity for GAGs ever observed for any chemokine.

Results and Discussion

Wild Type and Mutants CXCL12 Production

The CXCL12γ cDNA, obtained from Balb/C mouse brain mRNA was cloned and over expressed in E. coli, purified to homogeneity, and characterized by mass spectrometry, NMR and amino acid analysis. The preparation routinely yielded 4-5 mg of purified protein per liter of bacterial culture. Wild type and mutants CXCL12α, β and γ, (FIG. 9A) were also produced by chemical synthesis and characterized by ion spray mass spectrometry and HPLC analysis. Final purity of all samples was found to be, on average, in the range of 90-95%. The biological activity (chemotaxis) of the recombinant chemokine and its chemically synthesized homologue was identical (data not shown).

CXCL12γ has a Typical Chemokine Fold in the 1-68 Domain and an Unstructured C-Terminal Extension

CXCL12α structure has been solved both by X ray crystallography [25,26] and NMR spectroscopy [27]. The α and β isoform structures are similar [28] but no information has yet been reported for CXCL12γ. To perform structural and binding studies, recombinant CXCL12γ was purified from cells grown in ¹⁵NH₄Cl and ¹³C-glucose supplemented medium. Backbone resonances were assigned and the secondary structure content evaluated from ¹³C, ¹⁵N and ¹H frequencies (TALOS [29]). The fold similarity of CXCL12γ and α was assessed by recording orientational informations (N—H^{N} Residual Dipolar Couplings (RDC)) of partially aligned molecules in dilute liquid crystal [30], and NMR relaxation experiments were used to evaluate regions of flexibility. The first 68 residues of CXCL12γ have a spectrum very similar to that of CXCL12α [28,31], enabling the identification of most residues by visual inspection. This was confirmed by the complete assignment of CXCL12γ residues, but K1, E73 and K84 (FIG. 9B). Secondary structure prediction from the backbone chemical shifts indicated almost identical secondary structure content for CXCL12α and γ. Forty two N—H^{N} RDCs, in the 10-64 domain of CXCL12γ were analyzed against CXCL12α, 33 of them showed an overall good correlation (FIG. 9C), which suggests that CXCL12γ 1-68 domain and CXCL12α adopt the same tertiary structure. CXCL12γ 1-68 relaxation parameters (R1, R2 and ¹⁵_{N-}¹H NOes) were highly similar to those observed for monomeric CXCL12α [31] with the residues 10-64 being well structured. Residues 69-98 behaved differently: they were clustered between 8 and 8.5 ppm in the ¹H dimension, suggesting they were poorly ordered in solution (FIG. 9D). According to TALOS, only a few residues are predicted to adopt an extended conformation (FIG. 9A). Seven N—H^{N} RDCs were observed in the γ extension between 2 and 7 Hz, presumably indicative of averaged RDCs due to important flexibility. This domain, with negative ¹⁵N—¹H NOes and low R1 and R2 relaxation rates compared to the protein core, experienced fast timescale dynamics, confirming it was highly disordered in solution. Together, these data show that the C-terminal peptide is disordered and has no major effect on the structure of the first 68 residues of CXCL12γ. The prevalence of such non structured protein segments, recently became increasingly recognized [32]. These domains, known as intrinsically disordered or natively unfolded, usually feature a unique combination of low overall hydrophobicity and high net charge, a point that clearly characterize the CXCL12γ C-terminal peptide. Proteins with such disordered regions are believed to performed critical functions, including molecular recognition through large and accessible interaction surfaces. In view of the highly basic nature of the CXCL12γ C-terminal domain, its disordered state, and the importance of GAG recognition for chemokine function, we then investigated the ability of CXCL12γ to interact with a variety of GAGs, including heparin (HP) HS, and dermatan sulfate (DS), and compared it to that of CXCL12α, β, which C-termini are distinct.

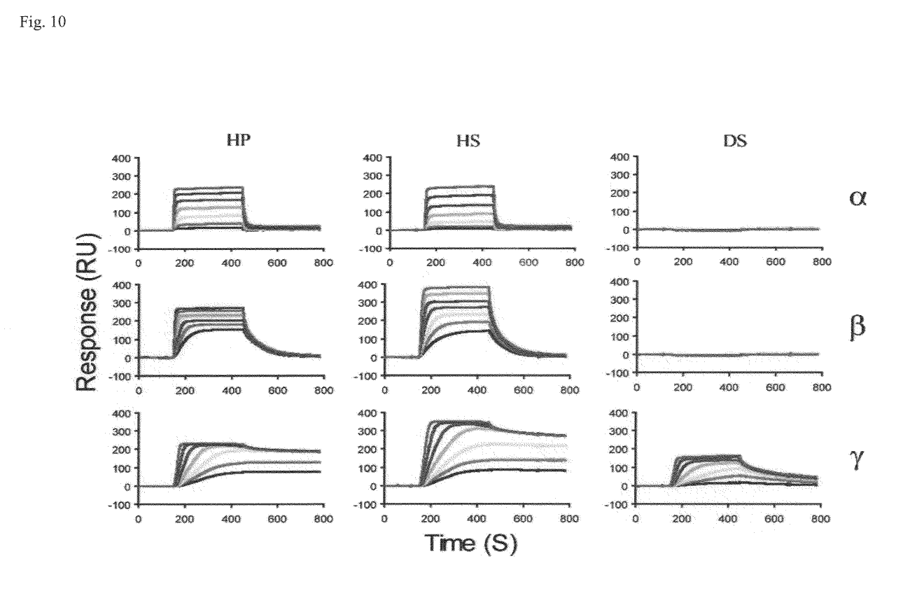

CXCL12α, β and γ differently bind to GAGs To determine the GAG binding ability of CXCL12α, β and γ isoforms we adopted a solid phase assay, in which reducing end biotinylated HP, HS or DS were captured on top of a streptavidin coated sensorchip, a system that mimics, to some extent, the cell membrane-anchored proteoglycans. Surface plasmon resonance (SPR)'s real time monitoring was exploited to measure changes in refractive index caused by the binding of chemokines to each of the immobilized GAGs. Binding curves, obtained when the CXCL12 isoforms were flowed over the HP, HS and DS surfaces, showed marked differences (FIG. 10). These experiments first indicated that while CXCL12γ interacts with HP, HS and DS, CXCL12α and β only recognize HP and HS, suggesting that the C-terminal domain, which characterizes the γ isoform, enables the chemokine to extend the range of GAGs to which it binds. Visual inspection of the sensorgrams also showed major differences during the dissociation phase. CXCL12α dissociated from the immobilized GAGs rapidly (binding curves returned to the base line within a minute), while CXCL12γ formed tight complexes, and CXCL12β displayed an intermediate behaviour. Preliminary analysis of the binding curves indicated that the binding rates were dominated by mass transfer, and global fitting of the binding curve returned values with low significances (see below). Because we generated data in which the association phase was allowed to proceed to equilibrium, affinity data were derived independently from the kinetic. By plotting R_{eq}/C against R_{eq} for different concentrations of chemokine (in which R_{eq} are the steady state values at equilibrium and C the concentrations of injected chemokine), straight lines were obtained (data not shown) which slopes, corresponding to the equilibrium constant Kd, are reported in Table I. These analyses demonstrate that CXCL12γ interacts with GAGs with an unprecedented capacity, representing a 2 log increase compared to CXCL12α, and suggesting a strong participation of the C-terminal domain in the binding reaction.

TABLE I
Equilibrium dissociation constant of CXCL12 for HP, HS and DS

| | HP | HS | DS |
|---|---|---|---|
| CXCL12 α | 93 ± 6.1 | 200 nM ± 14 | No binding |
| CXCL12 β | 24.7 nM ± 2.6 | 53 nM ± 2.7 | No binding |
| CXCL12 γ | 0.91 nM ± 0.07 | 1.5 nM ± 0.2 | 4.8 nM ± 0.04 |

The equilibrium levels of bound CXCL12 were extracted from the sensorgrams of FIG. 2 at the end of the association phases (apart from the lowest CXCL12 concentrations which in some cases did not reach equilibrium) and used to calculate the dissociation constant (Kd), using the Scatchard plot. Results are expressed in nM as means±SEM of 3 to 7 experiments

Heparin Derived Oligosaccharides Interact with CXCL12γ C-Terminal Domain and Reduce its Mobility

In view of the above data, which support the existence of additional GAG binding sites within the C-terminal domain of CXCL12γ, we performed titration experiments of ¹⁵N-CXCL12γ with different HP derived di-(dp2), tetra-(dp4) and octa-(dp8) saccharides. The CXCL12γ/oligosaccharide interactions were in fast exchange regime compared to NMR chemical shift timescale, typical of interactions in the μM-mM Kd range. Interaction with dp4 reached saturation, with an apparent Kd of about 250 μM.

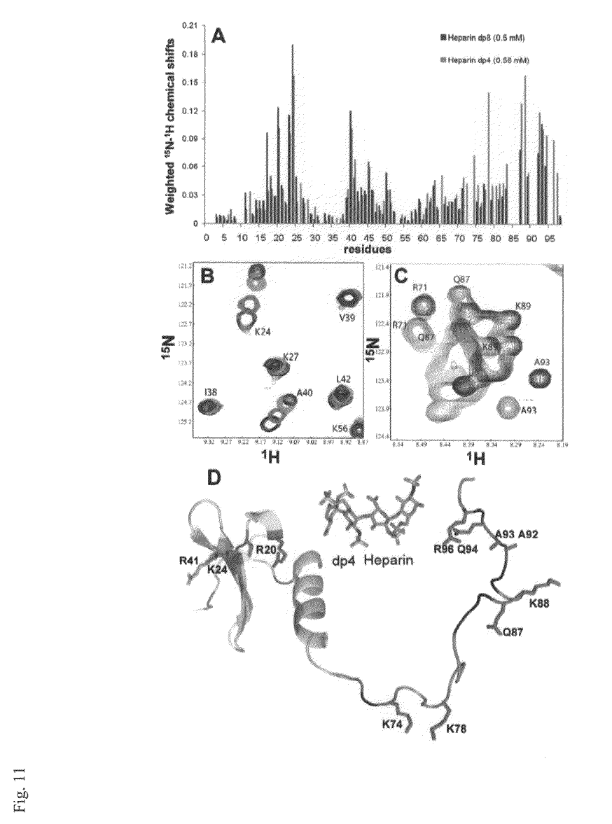

Several resonances in the γ extension were highly perturbed upon interaction (FIG. 11A), but could not be individually followed during titrations and backbone resonance assignment was performed on the ¹⁵N-¹³C-CXCL12γ/dp4 complex. Interactions of dp2, dp4 and dp8 with CXCL12γ revealed two binding domains on the protein (FIG. 11). On the CXCL12γ core region, the most perturbed residues form a continuous surface, from R20 to R41 (FIG. 11D), including V23, K24, A40, and N45. This binding surface suggested an oligosaccharide orientation more or less perpendicular to the β sheet that differs from the orientation of a dp12 in complex with a CXCL12α dimer, where the oligosaccharide also binds K24 and R41 but is aligned along the first β strand [24]. On the C-terminal extension, most of the residues were perturbed by the interaction in particular residues 83 to 97. Mab 6E9, which epitope consists of residues 78-80, still bound to the CXCL12γ/GAG complex (data not shown), further supporting the importance of the distal part of the C-terminus Backbone chemical shifts from CXCL12γ/dp4 complex did not reveal any secondary structural changes compared to the free protein, and no appearance of secondary structure elements in the C-terminal extension. ¹⁵N—¹H heteronuclear NOes on the complex (data not shown) indicated however a significant decrease in mobility upon dp4 binding for the γ extension with positive NOe values for residues 82 to 89. A maximum NOe value around 0.2 for Q87 (data not shown) suggested nevertheless that, even in complex with HP derived oligosaccharides, the γ extension still exhibits important flexibility.

The C-Terminal Domain and the Binding Sites in the Core Structure of CXCL12γ Differently Contribute to the Binding

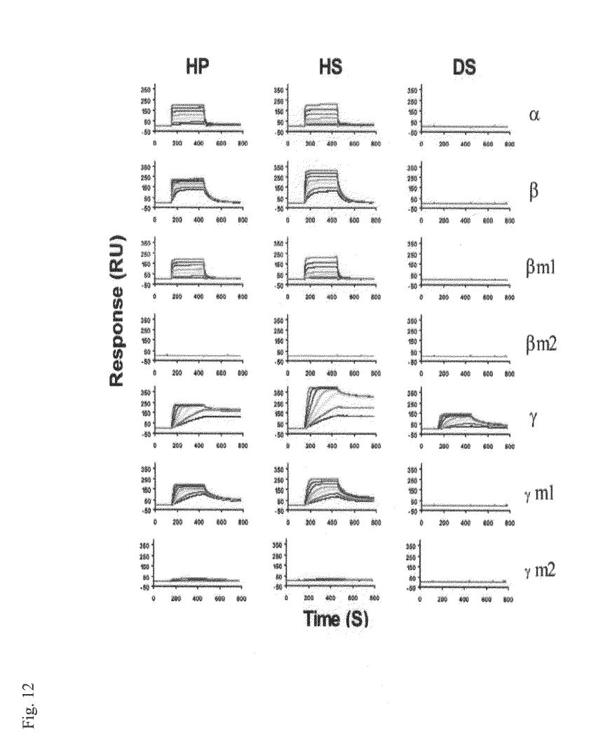

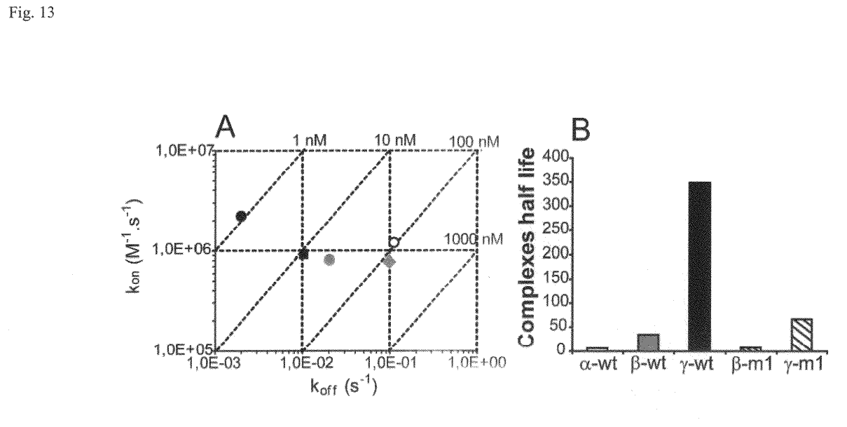

To further analyze the respective contributions in GAG recognition, of the core region and the C-terminal domain of CXCL12, mutations were introduced in both parts of the chemokine (see FIG. 9A) and their binding profiles were analyzed using the SPR assay (FIG. 12). As mentioned above, simultaneous fitting of the association and dissociation phases was not possible, presumably due to fast on rate which causes strong mass transport limitation during the association phase (data not shown), and possibly rapid rebinding of the dissociated molecules during the dissociation phase. The dissociation rates (k_{off}) were thus first measured at the beginning of the dissociation phase (where rebinding is limited because the number of free immobilized GAGs remains low) and the on rates (kₒₙ) were then calculated using the equilibrium dissociation constant (kₒₙ=k_{off}/Kd). Results are indicated in FIG. 13 and show that the C-terminal domain, while having limited effect on the on rate, essentially determines the velocity at which the formed complex dissociates. This is particularly marked for the γ isoform, which dissociates from HP with a k_{off} of 0.0019 M⁻¹s⁻¹ compared to 0.111 M⁻¹ s⁻¹ for CXCL12α and 0.0204 M⁻¹s⁻¹ for CXCL12β. In agreement with these observations, mutations of the 3 basic residues present at the C-terminus of the β isoform (β-m1) did not change the on rate, but increased the off rate to a value of 0.098 M⁻¹s⁻¹, thus resulting in a behavior very close to that of CXCL12α (FIG. 13), with an overall affinity of 125 nM for HP and 192 for HS (compare with results in Table I). Mutations in the core region (K24S/K27S), that in CXCL12α completely abolished HP/HS binding [24], were also introduced in β-m1 to give rise to a new mutant (β-m2; see FIG. 9) which, as expected, did not bind anymore to GAGs. Similarly, the effect on GAG binding of mutations introduced in the C-terminal domain of CXCL12γ was analyzed. Amongst the 18 basic residues of this domain, 9 were changed for Ser which destroyed the 3 typical HP binding clusters (FIG. 9A). Preliminary analysis performed with C-terminal synthetic peptides (residues 69-98) indicated that the wild type sequence required 0.88 M NaCl to be eluted from a HP affinity column, while the mutant peptide eluted at 0.28 M NaCl. This mutant peptide did not show any binding up to 200 nM using the SRP assay, demonstrating that these mutations very strongly decreased its binding capacity (data not shown). The GAG binding profile of the mutated full length chemokine (γ-m1, which includes these 9 mutations), was characterized. We observed, that this mutant did not bind anymore to DS, supporting the view that the broad GAG binding activity of the CXCL12γ isoform relied on the net charge of its C-terminal domain. As could have been anticipated, γ-m1 displayed an increased dissociation rate compared to the wild type chemokine (FIG. 13A), confirming the role of the C-terminal domain in the complex stability. The equilibrium dissociation constant for HP of this mutant was 10.4 nM (32 for HS). Thus, although this C-terminal domain by itself has a highly reduced binding capacity, the full length molecule still interacts quite strongly with HP and HS, suggesting a predominant role for the core domain. Consistently with this hypothesis, additional mutations in the core structure (γ-m2) dramatically decreased HP and HS binding, strongly supporting the critical importance of the core domain binding site for the interaction. Half live of the different CXCL12/HP complexes=(−ln [0.5]/k_{off}) were calculated, which indicated that while CXCL12α/HP complex was characterized by a half life of 6 seconds, CXCL12γ/HP complex was characterized by a half life of 350 seconds (FIG. 13B). Together, these data show that few key residues of the structured domain of CXCL12γ (in particular K24/27) constitute a strictly required binding site while, surprisingly in view of its high positive charge, the unfolded C-terminus appears to primarily functions in stabilizing the formed complex. Such different contributions between the two domains could be explained by the fact that electrostatic interactions are not always energetically positive. Favorable coulombic interactions formed in a final complex can be some times largely offset by the desolvation cost associated with the binding process [33], an effect that could occurs within the unfolded and largely solvent accessible C-terminus of CXCL12γDNA-binding domains frequently have N- or C-terminal extensions, enriched in basic residues, and disordered in solution. The contribution of such basic tails, which increase the affinity for target DNA, has been studied in the context of protein-DNA interaction [34], but to our knowledge this has not yet been described for protein-GAG complex. In any case, the present findings support the view that for CXCL12γ, a large and unstructured C-terminal domain functions as an accessory “binding cassette” which, in cooperation with a restricted and well defined binding site in the core structure provides very tight binding to GAGs.

CXCL12γ Displays Enhanced Binding to Cell Surface Expressed HS Compared to CXCL12α

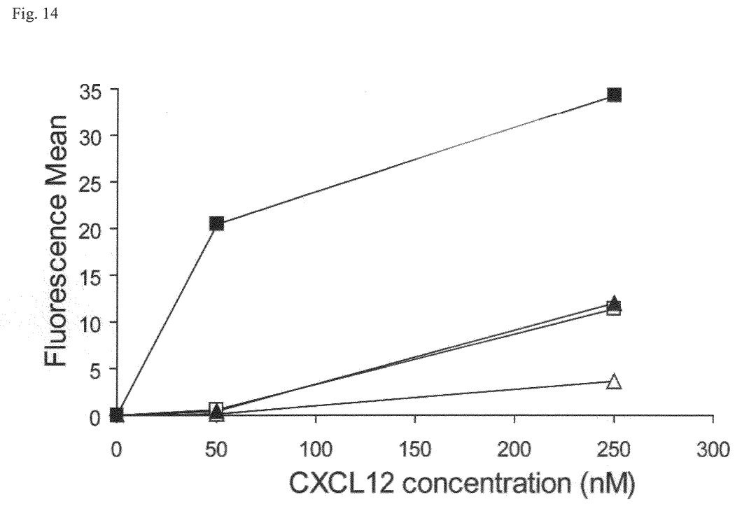

To investigate whether HS, in the context of the cell surface, also interacted more efficiently with CXCL12γ than with CXCL12α we then compared the adsorption of these two isoforms on CXCR4 negative CHO cells. Flow cytometry and mAb K15C, which recognize an epitope outside the HS binding site and present in all CXCL12 isoforms [35] were used for this purpose. Data are reported on FIG. 14, and show that binding to wild type CHO-K1 cells was greatly enhanced for CXCL12γ as compared to CXCL12α. In particular, at low concentration (50 nM), CXCL12α did not displayed significant binding, while the γ isoform bound strongly to the cell surface, a point that fits well with the Biacore data (FIG. 10).

These interactions were strongly reduced on HS deficient CHO-pgsD677 cells, demonstrating the importance of HS in the binding.

CXCL12γ displays reduced binging to- and signaling through-CXCR4 To analyze the binding of CXCL12γ to CXCR4, we set up an assay in which we compared the ability of the α and γ isoforms to compete with ¹²⁵I-labeled CXCL12α. This was done on T lymphoblastoid cell lines (CEM or A3.01) which does not express detectable amount of GAGs (data not shown) enabling the strict analysis of CXCL12/CXCR4 interaction. Results showed that CXCL12α and γ, although featuring identical receptor binding domain (localized in the N-terminus), behave differently, the latter having a reduced ability to bind to CXCR4 with an IC₅₀ of 350 nM versus 15 nM for CXCL12α. This difference clearly relied on the C-terminal domain of CXCL12γ, since specific mutations within this domain (γ-m1) restored binding to a level comparable to that of CXCL12α (FIG. 15A). In agreement with this observation, CXCL12γ has a reduced ability to stimulate intracellular calcium mobilization compared to the α isoform (FIG. 15B). The large amount of GAGs usually found at the cell surface, the reduced affinity of CXCL12γ for CXCR4 and its very high affinity for HS, suggest that within tissues the γ isoform might be predominantly in a bound form, associated to GAGs, and either stabilized to prevent proteolytic degradation and/or immobilized to allow continued and localized stimulation of cells.

Conclusion

The binding of proteins to GAGs is the prerequisite for a large number of cellular processes and regulatory events. The chemokine system, in particular, strongly depends on HS which are believed to ensure the correct positioning of chemokines within tissue. In this report, we described that a new splice variant of CXCL12, CXCL12γ, displayed an unusually high affinity for GAGs and investigated the structural determinants involved. The first 68 amino acids of the chemokine, common to all CXCL12 isoforms, displayed both the CXCR4 binding domain and a first, well defined, HS specific binding site. To this common platform is added by alternative spicing of the cxcl12 gene different peptides which contain a second GAG binding domain, limited to 4 additional residues for CXCL12β but as long as 30 residues for CXCL12γ. This domain, which remains unfolded, appeared to mainly function by stabilizing the chemokine/HS complex. This, in combination with the structured first HS binding site, provides the protein with an unprecedented high affinity for HS. Interestingly, it has been described that polypeptide segments generated by alternative splicing are mostly intrinsically disordered [36]. This has been thought as a way to generate functional diversity without structural modification or complication. Our present findings fit well with this proposed mode of action. Thus, by encoding a singular domain, bearing the CXCR4 binding site, on which is added distinct C-terminus, CXCL12 may display distinct regulatory functions. The observation that the different CXCL12 isoforms mostly differ by their ability to interact with GAGs, offers an unprecedented opportunity to ascertain the importance of chemokine/GAG bindings in the regulation of in vivo cell migration. Regarding CXCL12γ, the remarkable conservation, within mammals, of its entire c-terminal sequence is intriguing for a domain which essentially features electrostatic interactions, and argues in favor of an important role played by this isoform. The observation that GAGs trigger a rapid and almost irreversible accumulation of CXCL12γ suggests that within tissues it should exist essentially in a bound form in nearby cells, presumably to allow continued and localized cellular stimulation. These data are compatible with a selective role of this isoform, and indicate that GAGs could be critical in orchestrating the CXCL12 mediated oriented migration of cells, depending on the chemokine isoform and the nature of the GAGs to which it binds, either during development or postnatal life.

Materials and Methods

CXCL12 Production and Characterization

Murin CXCL12γ cDNA was inserted in a pET17b (Novagen) expression vector between NdeI and SpeI restriction sites, and checked by DNA sequencing. CXCL12γ was overexpressed overnight in E. coli BL21 (DE3) cells, with 0.4 mM IPTG, either in LB or M9 minimal medium supplemented with ¹⁵NH₄Cl and ¹²C or ¹³C-glucose for isotopic enrichment. After 30 minutes sonication at 4° C. in 50 mM Tris pH 8.0 (buffer A), inclusion bodies were pelleted (20000 g for 15 minutes) and washed with buffer A supplemented with 2M urea and 5% Triton X100, then with 2 M urea and finally with buffer A. Inclusion bodies were solubilised for 15 min at 50° C. in buffer A with 7.5 M GdCl₂ and 100 mM DTT. Refolding was performed by rapid dilution with buffer A up to 1 M GdCl₂. The mixture was gently stirred overnight at 4° C. after addition of Complete protease inhibitors (Roche), then diluted 4 times with buffer A and loaded onto a 3 ml Source S column (Amersham) equilibrated in 20 mM Na₂HPO₄ pH 6.0. CXCL12γ was eluted with a NaCl gradient, concentrated and further purified on a G75 gel filtration column (Amersham) in 20 mM Na₂HPO₄, 150 mM NaCl pH 6.0. Purified material was analyzed by MALDI mass spectrometry and quantified by amino acids analysis. Wild type and mutants CXCL12α, β and γ were also produced by chemical synthesis using the Merrifield solid-phase method and fluorenylmethyloxycarbonyl chemistry, as described [24].

Preparation of Heparin Derived Di- Tetra- and Octa-Saccharides

Porcine mucosal HP was depolymerized with heparinase I. The digestion mixture was resolved from di-(dp2) to octa-(dp18) decasaccharide, and dp2 to dp8 were further purified by strong-anion-exchange HPLC as described [37].

NMR Experiments

NMR experiments were recorded at 30° C. on Varian spectrometers (600 INOVA, 600 DD or 800 MHz with cryoprobe), processed with NMRpipe and analyzed with NMRview. CXCL12γ backbone assignment and relaxation experiments were recorded on 1 mM ¹⁵_{N-}¹³C sample in 20 mM NaH₂PO₄ pH 5.7, 10% D₂O, 0.01% NaN₃ with protease inhibitors at 600 MHz. HNCACB, CBCA(CO)NH and HNCO, ¹⁵N—¹H NOes and T₂ experiments were from Varian Biopack and T₁ experiment from [38]. Relaxation times were between 10 and 190 ms for T₂ and 10 and 180 ms for T₁. RDCs were measured as the difference between isotropic (25° C.) and anisotropic (34° C.) IPAP experiments [39] at 600 Mhz. 5% Bicelles (DMPC/DHPC 3:1 ratio) was used as the alignment medium with 180 μM of CXCL12γ in standard NMR buffer. The program MODULE was used to calculate the alignment tensor from the CXCL12α molecular shape and evaluate the correlations between experimental and backcalculated RDCs [40]. RDC data were evaluated against all CXCL12α published structures and fitted best 1 VMC monomeric NMR structure [41]. Residues with the lowest correlations with respect to backcalculated data (11, 19, 20, 23, 35, 45, 46, 48 and 63) were excluded from the fit (and the calculation of the alignment tensor). These outliers are located mostly within regions of structural heterogeneity between the different published structures of CXCL12α. Titration with HP derived oligosaccharides was performed with 200 μM ¹⁵N-CXCL12γ in the NMR buffer.

Surface Plasmon Resonance Based Binding Assay

Size defined HP (6 kDa), HS and DS were biotinylated at their reducing end, and immobilized on a Biacore sensorchip. For this purpose, flow cells of a CM4 sensorchip were functionalized with 2500 to 2800 resonance units (RU) of streptavidin as described [24] and biotinylated HP (5 μg/ml), HS (25 μg/ml) and DS (15 μg/ml) in HBS (10 mM HEPES, 150 mM NaCl, 3 mM EDTA, 0.005% surfactant P20, pH 7.4) were injected across the different flow cells to obtain immobilization levels of 40, 70 and 140 RU respectively. One flow cell was left untreated and served as negative control. For binding assays, 250 μl of CXCL12 were simultaneously injected, at a flow rate of 50 μl/min, across the control and the different GAG surfaces, after which the formed complexes were washed with running buffer for 5 min. The sensorchip surface was regenerated with a 3 minutes pulse of 2 M NaCl. Control sensorgrams were subtracted on line from GAG sensorgrams, and results analyzed using the Biaeval 3.1 software.

Binding of CXCL12 to CXCR4 and Cell Surface HS

CEM cells (10⁷ cells/ml) were incubated with 0.25 nM of ¹²⁵I-CXCL12α (Perkin-Elmer, 2200 Ci/mmol) and a range of concentrations of unlabelled CXCL12 (α, γ or γ-m1) in 100 μl of PBS for 1 h at 4° C. Incubations were stopped by centrifugation at 4° C. Cell pellets were washed twice in ice-cold PBS, and the associated radioactivity was counted. For measuring the ability of CXCL12 to interact with cellular HS, the CXCR4 negative CHO-K1 or HS-deficient CHO-pgsD677 (ATCC) were incubated with the

chemokine and after removal of unbound proteins, were labelled with an anti-CXCL12 mAb (clone K15C) and a PE-conjugated secondary antibody. Immunolabelled cells were analysed by flow cytometry using a FacsCalibur (BD Biosciences).

Intracellular Calcium Release Responses

Intracellular calcium measured in CXCR4-expressing cells loaded with fluo-4-AM (Interchim) was conducted in a Mithras LB 940 counter (Berthold Technologies). Briefly, A3.01 cells were incubated for 45 min at 37° C. in the load buffer (10 mM Hepes, 137.5 mM NaCl, 1.25 mM CaCl₂, 1.25 mM MgCl₂, 0.4 mM NaH2PO₄, 1 mM KCl, 1 mM Glucose) with 0.1% of pluronic acid and 0.5 mM of Fluo4-AM (10⁶ cells/mL). After a washing step, cells were suspended in load buffer at a final concentration of 2×10⁶ cells/mL and stored at 4° C. For intracellular calcium measurements, aliquots of cells (2×10⁵ cells) were preincubated at 37° C. for 1 min and then placed in a 96-well flat bottom plate. Fluorescence emission was recorded at 535 nM (excitation at 485 nM) every second before (basal fluorescence) and after programmed injection of different concentration of the ligands. Maximum and minimum fluorescence values were determined after addition of Triton X-100 and EDTA, respectively. Data are expressed as fluorescence increment rate after ligand addition.

### REFERENCES FOR EXAMPLE 2

1. Rossi D, Zlotnik A (2000) The biology of chemokines and their receptors. Annu Rev Immunol 18: 217-242.
2. Nagasawa T, Hirota S, Tachibana K, Takakura N, Nishikawa S, et al. (1996) Defects of B-cell lymphopoiesis and bone-marrow myelopoiesis in mice lacking the CXC chemokine PBSF/SDF-1. Nature 382: 635-638.
3. Agace W W, Amara A, Roberts A I, Pablos J L, Thelen S, et al. (2000) Constitutive expression of stromal derived factor-1 by mucosal epithelia and its role in HIV transmission and propagation. Curr Biol 10: 325-328.
4. Pablos J L, Amara A, Bouloc A, Santiago B, Caruz A, et al. (1999) Stromal-cell derived factor is expressed by dendritic cells and endothelium in human skin. Am J Pathol 155: 1577-1586.
5. Aiuti A, Webb I J, Bleul C, Springer T, Gutierrez-Ramos J C (1997) The chemokine SDF-1 is a chemoattractant for human CD34+ hematopoietic progenitor cells and provides a new mechanism to explain the mobilization of CD34+ progenitors to peripheral blood. J Exp Med 185: 111-120.
6. Bleul C C, Farzan M, Choe H, Parolin C, Clark-Lewis I, et al. (1996) The lymphocyte chemoattractant SDF-1 is a ligand for LESTR/fusin and blocks HIV-1 entry. Nature 382: 829833.
7. D'Apuzzo M, Rolink A, Loetscher M, Hoxie J A, Clark-Lewis I, et al. (1997) The chemokine SDF1, stromal cell-derived factor 1, attracts early stage B cell precursors via the chemokine receptor CXCR4. Eur J Immunol 27: 1788-1793.
8. Salcedo R, Oppenheim J J (2003) Role of chemokines in angiogenesis: CXCL12/SDF-1 and CXCR4 interaction, a key regulator of endothelial cell responses. Microcirculation 10: 359-370.
9. Zhu Y, Yu T, Zhang X C, Nagasawa T, Wu J Y, et al. (2002) Role of the chemokine SDF-1 as the meningeal attractant for embryonic cerebellar neurons. Nat Neurosci 5: 719-720.
10. Gerard C, Rollins B J (2001) Chemokines and disease. Nat Immunol 2: 108-115.
11. Orimo A, Gupta P B, Sgroi D C, Arenzana-Seisdedos F, Delaunay T, et al. (2005) Stromal fibroblasts present in invasive human breast carcinomas promote tumor growth and angiogenesis through elevated SDF-1/CXCL12 secretion. Cell 121: 335-348.
12. Oberlin E, Amara A, Bachelerie F, Bessia C, Virelizier J L, et al. (1996) The CXC chemokine SDF-1 is the ligand for LESTR/fusin and prevents infection by T-cell-line-adapted HIV-1. Nature 382: 833-835.
13. Balabanian K, Lagane B, Infantino S, Chow K Y, Harriague J, et al. (2005) The chemokine SDF1/CXCL12 binds to and signals through the orphan receptor RDC1 in T lymphocytes. J Biol Chem 280: 35760-35766.
14. Nagasawa T, Nakajima T, Tachibana K, Iizasa H, Bleul C C, et al. (1996) Molecular cloning and characterization of a murine pre-B-cell growth-stimulating factor/stromal cell-derived factor 1 receptor, a murine homolog of the human immunodeficiency virus 1 entry coreceptor fusin. Proc Natl Acad Sci USA 93: 14726-14729.
15. Gleichmann M, Gillen C, Czardybon M, Bosse F, Greiner-Petter R, et al. (2000) Cloning and characterization of SDF-1gamma, a novel SDF-1 chemokine transcript with developmentally regulated expression in the nervous system. Eur J Neurosci 12: 1857-1866.
16. Yu L, Cecil J, Peng S B, Schrementi J, Kovacevic S, et al. (2006) Identification and expression of novel isoforms of human stromal cell-derived factor 1. Gene 374: 174-179.
17. Lortat-Jacob H, Grosdidier A, Imberty A (2002) Structural diversity of heparan sulfate binding domains in chemokines. Proc Natl Acad Sci USA 99: 1229-1234.
18. Bernfield M, Gotte M, Park P W, Reizes O, Fitzgerald M L, et al. (1999) Functions of cell surface heparan sulfate proteoglycans. Annu Rev Biochem 68: 729-777.
19. Esko J D, Selleck S B (2002) Order out of chaos: assembly of ligand binding sites in heparan sulfate. Annu Rev Biochem 71: 435-471.
20. Campanella G S, Grimm J, Manice L A, Colvin R A, Medoff B D, et al. (2006) Oligomerization of CXCL10 is necessary for endothelial cell presentation and in vivo activity. J Immunol 177: 6991-6998.
21. Sadir R, Imberty A, Baleux F, Lortat-Jacob H (2004) Heparan sulfate/heparin oligosaccharides protect stromal cell-derived factor-1 (SDF-1)/CXCL12 against proteolysis induced by CD26/dipeptidyl peptidase IV. J Biol Chem 279: 43854-43860.
22. Handel T M, Johnson Z, Crown S E, Lau E K, Proudfoot A E (2005) Regulation of protein function by glycosaminoglycans—as exemplified by chemokines. Annu Rev Biochem 74: 385-410.
23. Sweeney E A, Lortat-Jacob H, Priestley G V, Nakamoto B, Papayannopoulou T (2002) Sulfated polysaccharides increase plasma levels of SDF-1 in monkeys and mice: involvement in mobilization of stem/progenitor cells. Blood 99: 44-51.
24. Sadir R, Baleux F, Grosdidier A, Imberty A, Lortat-Jacob H (2001) Characterization of the stromal cell-derived factor-1alpha-heparin complex. J Biol Chem 276: 8288-8296.
25. Dealwis C, Fernandez E J, Thompson D A, Simon R J, Siani M A, et al. (1998) Crystal structure of chemically synthesized [N33A] stromal cell-derived factor 1alpha, a potent ligand for the HIV-1 “fusin” coreceptor. Proc Natl Acad Sci USA 95: 6941-6946.
26. Ohnishi Y, Senda T, Nandhagopal N, Sugimoto K, Shioda T, et al. (2000) Crystal structure of recombinant native SDF-1alpha with additional mutagenesis studies: an attempt at a more comprehensive interpretation of accumulated structure-activity relationship data. J Interferon Cytokine Res 20: 691-700.
27. Crump M P, Gong J H, Loetscher P, Rajarathnam K, Amara A, et al. (1997) Solution structure and basis for functional activity of stromal cell-derived factor-1; dissociation of CXCR4 activation from binding and inhibition of HIV-1. Embo J 16: 6996-7007.
28. Veldkamp C T, Peterson F C, Pelzek A J, Volkman B F (2005) The monomer-dimer equilibrium of stromal cell-derived factor-1 (CXCL 12) is altered by pH, phosphate, sulfate, and heparin. Protein Sci 14: 1071-1081.
29. Cornilescu G, Delaglio F, Bax A (1999) Protein backbone angle restraints from searching a database for chemical shift and sequence homology. J Biomol NMR 13: 289-302.
30. Bax A (2003) Weak alignment offers new NMR opportunities to study protein structure and dynamics. Protein Sci 12: 1-16.
31. Baryshnikova O K, Sykes B D (2006) Backbone dynamics of SDF-1alpha determined by NMR: interpretation in the presence of monomer-dimer equilibrium. Protein Sci 15: 2568-2578.
32. Dyson H J, Wright P E (2005) Intrinsically unstructured proteins and their functions. Nat Rev Mol Cell Biol 6: 197-208.
33. Honig B, Nicholls A (1995) Classical electrostatics in biology and chemistry. Science 268: 11441149.
34. Crane-Robinson C, Dragan A I, Privalov P L (2006) The extended arms of DNA-binding domains: a tale of tails. Trends Biochem Sci 31: 547-552.
35. Amara A, Lorthioir O, Valenzuela A, Magerus A, Thelen M, et al. (1999) Stromal cell-derived factor-1alpha associates with heparan sulfates through the first beta-strand of the chemokine. J Biol Chem 274: 23916-23925.
36. Romero P R, Zaidi S, Fang Y Y, Uversky V N, Radivojac P, et al. (2006) Alternative splicing in concert with protein intrinsic disorder enables increased functional diversity in multicellular organisms. Proc Natl Acad Sci USA 103: 8390-8395.
37. Vanhaverbeke C, Simorre J P, Sadir R, Gans P, Lortat-Jacob H (2004) NMR characterization of the interaction between the C-terminal domain of interferon-gamma and heparin-derived oligosaccharides. Biochem J 384: 93-99.
38. Farrow N A, Muhandiram R, Singer A U, Pascal S M, Kay C M, et al. (1994) Backbone dynamics of a free and phosphopeptide-complexed Src homology 2 domain studied by 15N NMR relaxation. Biochemistry 33: 5984-6003.
39. Ottiger M, Delaglio F, Marquardt J L, Tjandra N, Bax A (1998) Measurement of dipolar couplings for methylene and methyl sites in weakly oriented macromolecules and their use in structure determination. J Magn Reson 134: 365-369.
40. Dosset P, Hus J C, Marion D, Blackledge M (2001) A novel interactive tool for rigid-body modeling of multi-domain macromolecules using residual dipolar couplings. J Biomol NMR 20: 223-231.
41. Gozansky E K, Louis J M, Caffrey M, Clore G M (2005) Mapping the binding of the N-terminal extracellular tail of the CXCR4 receptor to stromal cell-derived factor-1alpha. J Mol Biol 345: 651-658.

Obviously, numerous modifications and variations of the present invention are possible in light of the above teachings. It is therefore to be understood that within the scope of the appended claims, the invention may be practiced otherwise than as specifically described herein.

1. A composition comprising at least
a polypeptide sequence binding Glycosaminoglycans and/or fragments thereof; and
a molecule of interest.

2. A composition according to claim 1 wherein the polypeptide sequence binding Glycosaminoglycans is selected from the group consisting of CXCL2alpha CXCL2beta, and CXCL2gamma.

3. The composition according to claim 1, where the molecule of interest is a chemokine or a soluble protein.

4. The composition according to claim 3 wherein the chemokine is selected from the group consisting of VGEF, EGF, NGF, neurotrophins and FGF.

5. The composition of claim 2, wherein the homing molecules comprises at least a part of the C-terminal CXCL2gamma or a variant thereof.

6. A composition comprising a homing molecule and a molecule of interest, wherein the homing molecule comprises a polypeptide of formula [BBXB]n;
wherein B is a basic amino acid selected from the group consisting of arginine, lysine, and histidine, X is any other amino acid; and
n is an integer comprised between 2 and 5.

7. The compositions of claim 1, wherein the both molecules are in a simple association.

8. The compositions of claim 1, wherein both molecules are covalently associated.

9. The compositions of claim 1, wherein the composition is prepared by chemical covalent coupling, optionally including at least one spacer between the molecules or by genetic engineering by using hybrid polynucleotide sequences encoding for chimerical combined molecule, optionally including spacers.

10. A purified or chemically synthesized polypeptide of formula [BBXB]n wherein B is a basic amino acid selected from the group consisting of arginine, lysine, and histidine, X is any other amino acid and n is an integer from 2 to 5.

11. A purified polypeptide having the C-terminal fragment of the CXCL2gamma or a fragment containing at least 15 amino acids comprising at least two BBXB motifs.

12. The composition of claim 6, wherein the homing molecule comprises the amino sequence GRREEKVGKKEKIGKKKRQKKRKAAQKRKN (SEQ ID NO: 1) and/or a variant thereof comprising the core sequence that enables the haptotactic homing activity and having at least the amino acid sequence of at least two BBXB motifs and wherein the variant has a basic charge for the whole molecule.

13. An antibody directed to any one of the compositions and/or homing molecules of claim 1.

14. The antibody of claim 13, which is a polyclonal or monoclonal.

15-20. (canceled)

21. A method of testing comprising:
using a composition as a research tool, wherein the composition is selected from the group consisting of:
a composition comprising at least a polypeptide sequence binding Glycosaminoglycans and/or fragments thereof, and a molecule of interest;
a composition comprising a homing molecule and a molecule of interest, wherein the homing molecule comprises a polypeptide of formula [BBXB]n, B is a basic amino acid selected from the group consisting of arginine, lysine, and histidine, X is any other amino acid, and n is an integer comprised between 2 and 5;
a purified or chemically synthesized polypeptide of formula [BBXB]n, wherein B is a basic amino acid selected from the group consisting of arginine, lysine, and histidine, X is any other amino acid. and n is an integer from 2 to 5; and
a composition comprising a homing molecule and a molecule of interest, wherein the homing molecule comprises a polypeptide of formula [BBXB]n, B is a basic amino acid selected from the group consisting of arginine, lysine, and histidine, X is any other amino acid, and the homing molecule comprises the amino sequence GRREEKVGKKEKIGKKKRQKKRKAAQKRKN (SEQ ID NO: 1) and/or a variant thereof comprising the core sequence that enables the haptotactic homing activity and having at least the amino acid sequence of at least two BBXB motifs and wherein the variant has a basic charge for the whole molecule.

22. A hybridoma producing the antibody of claim 11.

23. A method of facilitating delivery of one or more therapeutic agents to a patient using a composition of claim 1.

24. The method of claim 23, which is used to treat a patient for occlusive arterial diseases and/or histological and functional regeneration of tissues (ie, epithelia, endothelia, muscle, connective, neuronal).

25. A method to in vivo immobilising or to concentrate a molecule of interest in a particular tissue environment of a host comprising the administration of a composition according to claim 1 to said host.

26. The method according to claim 25, wherein the in vivo immobilisation or concentration of the molecule of interest in a particular tissue environment improves the capacity to induce or regulate biological functions of said tissue.

27. A method to in vivo immobilising or to concentrate a molecule of interest in a particular tissue environment of a host comprising the administration of a composition according to claim 12 to said host.
